(19) United States
(12) Patent Application Publication
(10) Pub. No.: US 2014/0106015 A1
(43) Pub. Date: Apr. 17, 2014

(54) COMPOSITION CONTAINING HEAT-TREATED POWDER OR EXTRACT OF GLYCINE SOJA AS ACTIVE GRADIENT FOR PREVENTION AND TREATMENT OF DIABETES MELLITUS AND DIABETIC COMPLICATIONS

(71) Applicants: Dong Gyu Jang, Daejeon (KR); Wen Yi Jin, Daejeon (KR); Seog Mu Kim, Daejeon (KR)

(72) Inventors: Dong Gyu Jang, Daejeon (KR); Wen Yi Jin, Daejeon (KR); Seog Mu Kim, Daejeon (KR)

(73) Assignee: KOC BIOTEC CO LTD, DAEJEON (KR)

(21) Appl. No.: 14/110,619

(22) PCT Filed: Apr. 5, 2012
(86) PCT No.: PCT/KR12/02574

§ 371 Date: Dec. 24, 2013

(30) Foreign Application Priority Data
Apr. 8, 2011 (KR) 10-2011-0032805

(51) Int. Cl. A61K 36/48

(52) U.S. Cl. CPC A61K 36/48
USPC 424/757

(57) ABSTRACT

The present invention relates to a composition for the prevention and treatment of diabetes mellitus and diabetic complications. The composition includes as an active ingredient, a heat-treated powder or extract of glycine soja with hypoglycemic effects. The heat-treated powder and extract of glycine soja have outstanding hypoglycemic effects and therapeutic effects on diabetic complications. The composition of the present invention is useful as a pharmaceutical or food composition for the prevention and treatment (amelioration) of diabetes mellitus or diabetic complications.

### CROSS-REFERENCE TO RELATED APPLICATIONS

This application is a U.S. National Stage of International Application No. PCT/KR2012/002574, filed Apr. 5, 2012.

### BACKGROUND

1. Technical Field

The present invention relates to a composition for the prevention and treatment of diabetes mellitus and diabetic complications which contains, as an active ingredient, a heat-treated powder or extract of glycine soja with hypoglycemic effects.

2. Description of Related Art

Diabetes mellitus is a disease caused by deficiency of insulin secretion or diminished effectiveness of insulin. Diabetes mellitus happens when cells fails to use glucose properly in the body, resulting in hyperglycemia. Diabetes mellitus features the symptoms of hyperglycemia resulting from the dysregulation of physiological metabolisms such as carbohydrate, protein, lipid, and electrolyte metabolisms that is attributed to an imbalance of hormones including insulin. Persistent hyperglycemic symptoms cause blood flow disorders, retinal damage, neuronal cell damage, impaired renal functions, vascular complications, etc. and bring about serious chronic complications.

Particularly, cardiovascular diseases, such as arteriosclerosis, cerebral infarction, cerebral thrombosis, and myocardial infarction are more common in diabetic patients than in normal subjects (Fuller, J. H., Lancet, 1, pp. 1373-1376, 1980). Coronary diseases and cerebrovascular diseases are responsible for higher mortality of diabetic patients and are frequently developed by hypertension, hyperlipidemia, and obesity (HEO Gapbeom. The Korean Nutrition Society, Abstract Proceedings, pp. 15-18, 1984). 67% of patients with type 2 diabetes mellitus were reported to suffer from one or more lipid metabolic disorders (Harris, M. I. Diabetes Care, 23, pp. 754-758, 2000). The occurrence of such lipid metabolic disorders are associated with increased triglyceride level, elevated cholesterol level, and decreased HDL-cholesterol level (Goldberg, R. B. Diabetes Care, 4, pp. 561-572, 1981) and is a cause of coronary diseases as diabetic complications (Reaven, J. W. Am. J. Med., 83, pp. 31-40, 1987).

Diabetes mellitus is defined as a metabolic disorder induced by defect of insulin secretion from pancreatic cells. Diabetes mellitus is accompanied by excess production of glucose, degradation of body fats, and waste of proteins, and results in metabolic disturbance by abnormally stimulating the secretion of glucagon (Abrams, J. J., Ginsberg, H, et al., Diabetes, 31, pp. 903-910, 1982).

Diabetes mellitus is characterized by two types, i.e. type 1 diabetes mellitus and type 2 diabetes mellitus. Type 1 diabetes mellitus is caused by the deficiency of insulin, a hormone modulating blood glucose level. Type 1 diabetes mellitus is also called “juvenile diabetes” because it commonly occurs in teenagers and young adults in their twenties. Type 2 diabetes mellitus normally occurs in people over their forties and accounts for most of the diabetic population in Korea. Although the exact cause of type 2 diabetes mellitus is clearly unknown, environmental factors as well as genetic factors are known to be involved in the development of type 2 diabetes mellitus. Disorders of insulin secretion from pancreatic beta cells and defects of insulin action (insulin resistance) in target cells are observed as etiology of type 2 diabetes mellitus.

The most important goal in the treatment of diabetes mellitus is to keep the level of blood glucose as close as possible to the normal level. The regulation of postprandial blood glucose level as well as fasting blood glucose level is important in ameliorating diabetic symptoms and preventing and treating diabetic complications. Methods for treating diabetes mellitus include medicinal therapy, dietary therapy, and exercise therapy.

Alpha-glucosidase inhibitors, sulfonylurea agents, and biguanide agents are currently in use as oral hypoglycemic agents for type 1 and type 2 diabetes mellitus patients. Alpha-glucosidase inhibitors delay the digestion and absorption of dietary carbohydrates to prevent increases in postprandial blood glucose level and blood insulin level, achieving therapeutic effects on diabetes mellitus. Alpha-glucosidase inhibitors stimulate the secretion of insulin without causing hyperinsulinemia or hypoglycemia and promote the secretion of glucagon-like peptide-1, an inhibitor of glucagon secretion, in the small intestine (Mooradian, A. D., Thurman, J. E. et al., Drugs, 57, pp. 19-29, 1999; Baron. A. D. et al., Diabetes Research and Clinical Practice, 40, pp. S54-S55, 1998). Despite these advantages, long-term administration of alpha-glucosidase inhibitors may cause side effects such as abdominal inflation, vomiting, and diarrhea in some patients, limiting their use (Hanefeld, M. et al., Journal of Diabetes and its Complications, 12, pp. 228-237, 1998). Acarbose, voglibose, and miglitol are alpha-glucosidase inhibitors that are currently used in clinical applications. Sulfonylurea agents act on the human body to secrete insulin, help the human body respond to insulin, and prevent excretion of glucose from the liver into the blood, thus lowering the blood glucose level. Sulfonylurea agents were reported to cause side effects such as gastrointestinal tract disorders, undesirable skin responses, and body weight gain. The gastrointestinal tract disorders include constipation, diarrhea, nausea, and vomiting, and the skin responses include itchiness and rash. For example, glimepiride (Amaryl™), glipizide (Glucotrol™), and gliburide (Diabeta™) are drugs belonging to a group of sulfonylurea agents. An example of currently commercially available biguanide agents is metformin (Glucophage™). Biguanide agents allow the liver to more slowly excrete glucose stored therein and help the human body respond to insulin to keep blood glucose at a constant level. Biguanide agents were reported to cause side effects such as nausea, abdominal inflation, boredom, diarrhea, and anorexia.

Glycine soja Siebold & Zucc., simply glycine soja, is an annual climbing plant belonging to the Zingiberaceae family. Glycine soja grows to a height of about 2 meters. The hairs of Glycine soja are brown and rough as a whole. The leaves of glycine soja are arranged alternately on the stems and have long trifoliate stalks. The small leaves are oval lance-shaped with an obtuse tip, are 3-8 cm long, and have even edges. The stipules of Glycine soja are broad lance-shaped. Glycine soja blooms in July and August. The flowers are purple or red and grow on 2-5 cm long racemes. Glycine soja has five bell-shaped, hairy sepals, and butterfly-shaped corollas. The flower of Glycine soja has 10 stamens, each of which is split into two. The fruits of Glycine soja are 2-3 cm long, very hairy, and similar to bean pods. The seeds of glycine soja are oval or kidney-shaped and slightly flat (LEE Youngno. Colored Illustrated Guide to Korean Flora. Gyohaksa Co. Ltd., p 403, 1998). Glycine soja is also called “gaengmidu” or “nokgwak” as another name and is called “yadaedudeong” or “yaryodu” as a crude drug name (AHN Deokgyun, Illustrated Book of Korean Medicinal Herbs, Gyohaksa, p. 728, 2000). Glycine soja is considered the ancestor of Glycine max. Glycine soja is currently recognized as an edible plant but is not substantially used as a food material in actual cases. In most cases, glycine soja grows in nature. Glycine soja is often cultivated for genetic studies, such as genetic modification, due to its strong genes.

In connection with antidiabetic effects of plants in the Zingiberaceae family, Korean Patent Publication No. 10-2006-0107183 discloses antidiabetic effects such as hypoglycemic effects of a powder or extract of Rhynchosia nulubilis or vinegar-fermented Rhynchosia nulubilis, which is prepared by soaking and pickling Rhynchosia nulubilis in vinegar for about 10 days. Rhynchosia nulubilis is a perennial vine in the Zingiberaceae family and is also called “rat-eye bean” or “yeodu” as another name. Rhynchosia nulubilis has been used as a drug material due its good medicinal properties. Particularly, it is known that Rhynchosia nulubilis is effective in treating renal diseases, is good for blood circulation, has a detoxification function, and is used for the treatment of diseases symptomized by thirst. Rhynchosia nulubilis is also described as a herbal medicine in the literature, including “Bonchogangmok,” a book of ancient oriental medicine. Rhynchosia nulubilis is easily distinguishable from glycine soja in appearance due to its larger size than glycine soja. Rhynchosia nulubilis is mainly used at present as an edible material, unlike glycine soja.

Korean Patent Publication No. 10-2009-004503 discloses a pharmaceutical composition for the prevention and treatment of diabetes mellitus including, as active ingredients, anthocyanins extracted from the hull of glycine max. This patent publication describes that the anthocyanins extracted from glycine max reduce the level of glucose in the body or inhibit apoptosis of pancreatic cells, thus being effective in preventing or treating diabetes mellitus. Korean Patent Publication No. 10-2010-0127728 describes that an extract obtained by extracting beans with a lower alcohol at a low concentration, or a fraction of the extract improves blood circulation, ameliorates obesity, and is effective in preventing, ameliorating or treating diabetes mellitus, hyperglycemia and symptoms thereof. Korean Patent Publication No. 10-2006-0107183 describes that a powder or extract of Rhynchosia Nulubilis or vinegar-fermented Rhynchosia nulubilis has high insulin sensitivity in diabetes-induced experimental mice, achieving enhanced dietary availability, hypoglycemic effects, and weight loss of organs. EP 2172206 discloses a method for obtaining a sequoyitol-containing extract from a plant in the Zingiberaceae family. The extract has therapeutic effects on diabetes mellitus. However, the antidiabetic effects of the plants in the Zingiberaceae family disclosed in the prior art documents are simply based on the presence of anthocyanins or sequoyitol and inherent hypoglycemic effects of the bean extracts are not significant or are negligible.

### SUMMARY OF THE INVENTION

The present invention is intended to provide a new pharmaceutical composition and a food material for the prevention and treatment of diabetes mellitus that have superior hypoglycemic actions without causing side effects in diabetic patients. Particularly, the present inventors have unexpectedly found that antidiabetic effects of glycine soja are apparently distinguished from those of other plants in the Zingiberaceae family, and finally arrived at the present invention. The present invention is aimed at providing a steamed powder and an extract of glycine soja with superior antidiabetic effects that can prevent and treat diabetes mellitus in a safe and effective manner. For this aim, the present inventors conducted a series of experiments using db/db mouse models to identify pharmacological effects of the steamed powder and extract of glycine soja.

It is an object of the present invention to provide a pharmaceutical composition for preventing and treating diabetes mellitus and diabetic complications containing, as an active ingredient, a steamed powder or extract of glycine soja with outstanding hypoglycemic effects.

It is another object of the present invention to provide a food composition for preventing and ameliorating diabetes mellitus and diabetic complications containing, as an active ingredient, a steamed powder or extract of glycine soja with outstanding hypoglycemic effects.

According to one aspect of the present invention, there is provided a pharmaceutical composition for the prevention and treatment of diabetes mellitus or diabetic complications containing a heat-treated powder or extract of glycine soja as an active ingredient.

The glycine soja extract is intended to include a fraction obtained by fractionation of the extract with water or an organic solvent having 1 to 4 carbon atoms.

The diabetic complications include arteriosclerosis, cerebral infarction, cerebral thrombosis, myocardial infarction, hypertension, hyperlipidemia, and obesity.

According to another aspect of the present invention, there is provided a food composition for the prevention and amelioration of diabetic complications containing a heat-treated powder or extract of glycine soja as an active ingredient. The food is intended to include health functional foods, particularly, one whose formulation is selected from tablets, capsules, powders, granules, liquids, and pills. The food may be selected from beverages, powdered beverages, solid foods, chewing gums, teas, vitamin complexes, and food additives.

The streamed powder and extract of glycine soja has outstanding therapeutic effects on diabetic complications, such as amelioration of lipid metabolic disorders, together with outstanding hypoglycemic effects, which was identified through a series of experiments, including measurements of blood glucose, serum triglyceride and total cholesterol levels, using db/db mouse models in the present invention. The antidiabetic effects of the glycine soja streamed powder and extract are much superior to those of other plants in the Zingiberaceae family. Therefore, the pharmaceutical composition and the food composition of the present invention, each including the glycine soja streamed powder or extract as an active ingredient, are effective in preventing and treating (or ameliorating) diabetes mellitus or diabetic complications.

### BRIEF DESCRIPTION OF THE DRAWINGS

FIG. 1 shows the levels of glucose in blood samples drawn from the tail artery of each experimental group once a week during oral administration of drugs (test materials: DC 2 g/kg, banaba leaf extract 100 mg/kg, physiological saline) for 3 weeks (values are mean±SEM and values with different letters are significantly different from each other in each time point studied; statistically significant value compared with negative control by T test (**p<0.01, ***p<0.001));

FIG. 2 shows serum glucose levels measured after experiment was completed at the age of 9 weeks (*p<0.01);

FIG. 3 shows serum triglyceride (TG) levels measured after experiment was completed at the age of 9 weeks (*p<0.01);

FIG. 4 shows the levels of glucose in blood samples drawn from the tail vein of each experimental group once a week during oral administration of drugs (test materials) for 6 weeks (**p<0.01, ***p<0.01);

FIG. 5 shows serum glucose levels measured after experiment was completed at the age of 10 weeks (*p<0.01);

FIG. 6 shows serum triglyceride (TG) levels measured after experiment was completed at the age of 10 weeks (*p<0.01);

FIG. 7 shows serum total cholesterol levels measured after experiment was completed at the age of 10 weeks (*p<0.01);

FIG. 8 shows serum low-density lipoprotein cholesterol levels measured after experiment was completed at the age of 10 weeks (*p<0.01);

FIG. 9 shows serum high-density lipoprotein cholesterol levels measured after experiment was completed at the age of 10 weeks (*p<0.01);

FIG. 10 shows serum alanine aminotransferase (ALT) levels and serum aspartate aminotransferase (AST) levels measured after experiment was completed at the age of 10 weeks (*p<0.01);

FIG. 11 shows serum insulin levels measured after experiment was completed at the age of 10 weeks (*p<0.01);

FIG. 12 shows the total weights of abdominal, epididymal and inguinal adipose tissues excised from db/db mice after experiment was completed at the age of 10 weeks (*p<0.01);

FIG. 13 shows the levels of glucose in blood samples drawn from the tail vein of each experimental group once a week during oral administration of drugs (test materials) for 5 weeks (**p<0.01, ***p<0.001);

FIG. 14 shows serum triglyceride (TG) levels measured after experiment was completed at the age of 9 weeks (**p<0.01, ***p<0.001);

FIG. 15 shows serum total cholesterol levels measured after experiment was completed at the age of 9 weeks (**p<0.01, ***p<0.01):

FIG. 16 shows serum low-density lipoprotein cholesterol levels and high-density lipoprotein cholesterol levels measured after experiment was completed at the age of 9 weeks (**p<0.01, ***p<0.001);

FIG. 17 shows serum ALT levels and serum AST levels measured after experiment was completed at the age of 9 weeks (**p<0.01, ***p<0.001);

FIG. 18 shows serum insulin levels measured after experiment was completed at the age of 9 weeks (**p<0.01, ***p<0.01);

FIG. 19 shows the total weights of abdominal, epididymal and inguinal adipose tissues excised from db/db mice after experiment was completed at the age of 10 weeks (**p<0.01. ***p<0.001);

FIG. 20 shows the levels of glucose in blood samples drawn from the tail vein of each experimental group once a week during oral administration of drugs (test materials) for 5 weeks (**p<0.01 ***p<0.001);

FIG. 21 shows serum glucose levels measured after experiment was completed at the age of 9 weeks:

FIG. 22 shows serum triglyceride (TG) levels measured after experiment was completed at the age of 9 weeks (**p<0.01, ***p<0.001):

FIG. 23 shows serum total cholesterol levels measured after experiment was completed at the age of 9 weeks (**p<0.01, ***p<0.001);

FIG. 24 shows serum low-density lipoprotein cholesterol levels and high-density lipoprotein cholesterol levels measured after experiment was completed at the age of 9 weeks (**p<0.01, ***p<0.001);

FIG. 25 shows serum ALT levels and serum AST levels measured after experiment was completed at the age of 9 weeks (**p<0.01, ***p<0.001):

FIG. 26 shows serum insulin levels measured after experiment was completed at the age of 9 weeks (**p<0.01, ***p<0.001):

FIG. 27 shows the total weights of abdominal, epididymal and inguinal adipose tissues excised from db/db mice after experiment was completed at the age of 10 weeks (**p<0.01. ***p<0.001);

FIG. 28 shows the total weights of liver tissues excised from db/db mice after experiment was completed at the age of 10 weeks (**p<0.01, ***p<0.001);

FIG. 29 shows the levels of glucose in blood samples drawn from the tail vein of each experimental group once a week during oral administration of drugs (test materials) for 6 weeks (**p<0.01, ***p<0.001);

FIG. 30 shows the body weights of experimental groups measured once a week during oral administration of drugs (test materials) for 6 weeks;

FIG. 31 shows serum glucose levels measured after experiment was completed at the age of 9 weeks (**p<0.01, ***p<0.001);

FIG. 32 shows serum total cholesterol levels measured after experiment was completed at the age of 9 weeks (**p<0.01, ***p<0.001);

FIG. 33 shows serum low-density lipoprotein cholesterol levels and high-density lipoprotein cholesterol levels measured after experiment was completed at the age of 9 weeks (**p<0.01, ***p<0.001);

FIG. 34 shows serum BUN levels measured after experiment was completed at the age of 9 weeks (**p<0.01, ***p<0.001);

FIG. 35 shows serum insulin levels measured after experiment was completed at the age of 9 weeks (**p<0.01, ***p<0.001);

FIG. 36 shows the total weights of abdominal, epididymal and inguinal adipose tissues excised from db/db mice after experiment was completed at the age of 10 weeks (**p<0.01, **p<0.001);

FIG. 37 shows the total weights of liver tissues excised from db/db mice after experiment was completed at the age of 10 weeks (**p<0.01, ***p<0.001); and

FIGS. 38 to 40 show the results of analysis for active ingredients in DC60-1, DC60-2, DC5, and DC25.

### DETAILED DESCRIPTION OF PREFERRED EMBODIMENTS

The present invention provides a pharmaceutical composition or food composition for preventing and treating (ameliorating) diabetes mellitus and diabetic complications containing a heat-treated powder or extract of glycine soja as an active ingredient.

The diabetic complications mean diabetes-related diseases, and examples thereof include, but are not limited to, particularly, arteriosclerosis, cerebral infarction, cerebral thrombosis, myocardial infarction, hypertension, hyperlipidemia, and obesity.

As defined herein, “glycine soja,” whose scientific name is Glycine soja Siebold & Zucc., is an annual climbing plant belonging to the Zingiberaceae family. Glycine soja is called “gaengmidu” or “nokgwak” as another name and is called “yadaedudeong” or “yaryodu” as a crude drug name. The fruits of glycine soja are 2-3 cm long, very hairy, and similar to bean pods. The seeds of glycine soja are oval or kidney-shaped and slightly flat. The term “glycine soja” used herein is intended to include its seeds. The term “glycine soja powder” used herein means a dry powder of glycine soja seeds without being steamed. Naturally growing or cultivated glycine soja is available in the present invention. The term “heat-treated powder” of glycine soja used herein means a powder obtained by heat treating glycine soja or a glycine soja powder at 100° C. or less. The term “streamed powder” of glycine soja used herein means a powder obtained by steaming glycine soja or a glycine soja powder at 100° C. or less.

The heat-treated powder of glycine soja as an active ingredient of the pharmaceutical composition or food composition according to the present invention is preferably obtained by heat treating a glycine soja powder at 40 to 100° C., more preferably by steaming a glycine soja powder at 60 to 90° C.

The glycine soja extract as an active ingredient of the composition according to the present invention is obtained by extracting a glycine soja powder with a suitable solvent at a particular temperature, preferably 100° C. or less, more preferably 0 to 100° C., particularly preferably 0 to 90° C. The solvent is used in an amount about 2 to about 15 times, preferably about 5 to about 10 times greater than that of the sample. The solvent is preferably water, an organic solvent having 1 to 4 carbon atoms, or a mixture thereof. Particularly preferred is a polar solvent selected from water. C₁-C₄ lower alcohols (for example, methanol, ethanol, propanol, and butanol), and mixtures thereof. The extraction may be performed by a suitable technique known in the art, for example, hot-water extraction, cold dipping extraction, reflux cooling extraction or ultrasonic extraction. Preferably, the glycine soja extract is obtained by extracting a glycine soja powder with hot water at 90° C. or less, followed by filtration under reduced pressure and concentration. The glycine soja extract is intended to include a fraction obtained by fractionation of the extract with water or an organic solvent having 1 to 4 carbon atoms. The fractionation of the glycine soja extract may be performed by a suitable technique known in the art (Harborne J. B. Phytochemical methods: A guide to modern techniques of plant analysis, 3rd Ed., pp. 6-7, 1998).

The glycine soja powder may be prepared by harvesting naturally growing or cultivated glycine soja, drying the glycine soja using a general drying technique, and grinding the dry glycine soja using a pulverizer. The glycine soja powder is intended to include its freeze-dried form.

In the present invention, therapeutic effects of the glycine soja steamed powder and extract on diabetes mellitus and diabetic complications were identified through a series of experiments using db/db mouse models. In the following Examples Section, including experimental examples, anti-diabetic effects of the glycine soja steamed powder and extract in BKS.Cg-m^{+/+} Lepre^{db/J} mice as type 2 diabetes mellitus animal models were confirmed by modifications of the methods disclosed in the literature [Am. J. Physiol. Endocrinol. Metab. 288: E510-E518 (2005); Diabetologia. 49: 1647-1655 (2006); J. Ethnopharmacol. 103: 491-495 (2006); J. Med. Chem. 43: 3487-3494 (2003); Metabolism 50: 1049-1053 (2001); Nutrition & Metabolism 53: 488-499 (2004)].

First, therapeutic effects of the glycine soja steamed powder on type 2 diabetes mellitus and diabetic complications were confirmed using banaba leaf as positive control [Experimental Example 1]. Recent research has revealed that banaba leaf has efficacy on type 2 diabetes mellitus. Banaba (Lagerstroemia speciosa Pers.) is a perennial evergreen tree growing naturally in tropical, sub-tropical area. Banaba leaves include corosolic acid, zinc, iron, calcium, and magnesium as major ingredients. The content of corosolic acid as an active ingredient in banaba leaves may vary. Corosolic acid is on average present in an amount of about 0.1 to about 0.35%. Recent studies have confirmed that corosolic acid functions to rapidly absorb glucose into cells, i.e. to activate a glucose transporter, and thus acts to suppress an increase in blood glucose level without affecting hypoglycemic action and normal blood glucose level. That is, corosolic acid has the same function as insulin.

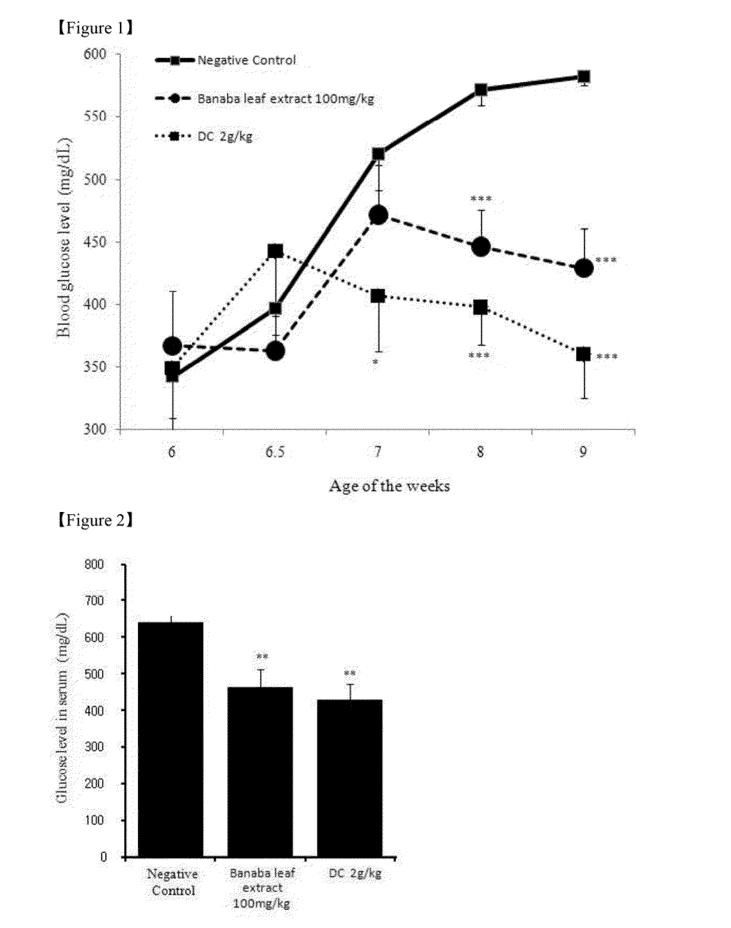

6-week-old db/db mouse models were orally administered 100 mg/kg of a banaba leaf extract as positive control, 2 g/kg of the glycine soja streamed powder (DC) as test group, and the same amount of physiological saline as negative control (NC) twice (in the morning and afternoon) a day for 3 weeks. The results are shown in FIG. 1. As shown in FIG. 1, the blood glucose level of the negative control (NC) increased to 582.1 mg/dL at the age of 9 weeks, which was higher by 70% or more than that (342.1 mg/dL) at the age of 6 weeks. In contrast, the blood glucose level of the DC-administered group was greatly suppressed compared to that of the non-administered group. The blood glucose level of the DC-administered group was 360.5 mg/kg at the age of 9 weeks, which corresponds to a 37.9% decrease compared to that of the non-administered group. After the experiment was completed at the age of 9 weeks, the serum glucose level of the non-administered group (NC) was 641 mg/dL and that of the DC-administered group was 427.0 mg/dL. The DC-administered group showed a significant decrease (33.4%) in serum glucose level compared with the non-administered group (FIG. 2).

Hyperlipidemia results from lipid metabolic disorders, which are commonly observed in patients with type 2 diabetes mellitus. The most frequent hyperlipidemia is hypertriglyceridemia. An increase in blood triglyceride (TG) level promotes insulin resistance to make the regulation of blood glucose more difficult, causing the development of arteriosclerosis. After completion of the experiment at the age of 9 weeks, serum samples were separated and serum triglyceride (TG) levels were measured. The serum triglyceride (TG) level of the non-administered group was 67.0 mg/dL and that of the DC-administered group was 37.0 mg/dL. The DC-administered group showed a statistically significant decrease (44.7% or more) in serum triglyceride level compared with the NC (p<0.001).

From the above experimental results, it could be confirmed that the glycine soja streamed powder (DC) significantly decreased the levels of blood glucose and triglyceride in the db/db mice compared with the negative control, and showed much greater decrements in blood glucose and triglyceride levels than the banaba leaf extract as positive control. Particularly, the glycine soja streamed powder (DC) was observed to have hypoglycemic efficacy to ameliorate fasting hyperglycemia. An acute toxicity experiment revealed that the DC is a safe drug and food material.

Metformin is most widely used at present for diabetes mellitus treatment. In the present invention, the glycine soja streamed powder and metformin were tested to compare their antidiabetic efficacies. The test results demonstrated better efficacy of the glycine soja streamed powder [Experimental Example 2]. Metformin is known to inhibit gluconeogeneic enzymes to reduce the production of glucose in the liver. In this experiment, changes in blood glucose level were measured during administration of the glycine soja streamed powder (DC) and metformin as positive control for 6 weeks. As a result, the blood glucose levels of the DC-administered group and the metformin-administered group were suppressed compared to the blood glucose level of the non-administered group (NC). The DC was found to have superior suppressive effects on blood glucose compared to metformin. The blood glucose levels of the DC-administered group and the non-administered group (NC) measured at the age of 10 weeks were 240.5 mg/dL and 545.8 mg/dL, respectively. That is, the DC-administered group showed a statistically significant decrease (55.9% or more) in blood glucose level compared with the non-administered group (NC).

One of the main effects of metformin is to enhance insulin sensitivity. The major mechanism by which insulin sensitivity is enhanced by metformin is associated with a reduction in endogenous glucose production, particularly, gluconeogenesis. Metformin is also known to lower the level of free fatty acids. The research results so far indicate that metformin significantly lowers the levels of total cholesterol and LDL cholesterol in type 2 diabetes mellitus patients with dyslipidemia. In addition, the trend of lowering triglyceride (TG) level and increasing HDL cholesterol level is observed in metformin despite its statistical insignificance. Also in the experiments of the present invention, the levels of total cholesterol, LDL-cholesterol, and triglyceride were considerably decreased in the metformin-administered group compared to in the non-administered group (NC). The DC administration showed a tendency to statistically significantly decrease the levels of total cholesterol, LDL-cholesterol, and triglyceride compared with the metformin administration (FIGS. 6 to 8). These results suggest outstanding effects of the DC on blood circulation.

In another experiment of the present invention, the serum insulin level of the non-administered group (NC) was 5.72 ng/ml and that of the DC-administered group was 4.07 mg/dL, which was lower by 28.8% than that of the non-administered group, but there was no statistical significance between the two groups. The serum insulin levels of the metformin-administered group and a bean powder-administered group were also slightly lower than the serum insulin level of the non-administered group, but there were no statistical significances among the groups. These results suggest that, similarly to metformin, the DC has suppressive efficacy on blood glucose and ameliorating efficacy on insulin resistance, which is a problem of type 2 diabetes mellitus.

These experimental results confirmed that the glycine soja streamed powder (DC) significantly decreased the levels of blood glucose, triglyceride, total cholesterol, and LDL cholesterol in db/db mice compared to the positive control (metformin) and the negative control. Particularly, the DC was observed to have outstanding effects on blood circulation such as hypoglycemic efficacy to ameliorate fasting hyperglycemia and efficacy to ameliorate insulin resistance.

During oral administration of the glycine soja powder (1.5 g/kg/day), and a water extract (300 mg/kg/day), a methanol extract (300 mg/kg/day), a hexane fraction (100 mg/kg/day), a butanol fraction (100 mg/kg/day), an ethyl acetate fraction (100 mg/kg/day), and a water fraction (100 mg/kg/day) of glycine soja to 4-week-old db/db mouse models for 5 weeks, hypoglycemic effects of the test materials were measured [Experimental Example 3]. As a result, the blood glucose levels of all test groups were statistically significantly lower than those of the control groups (at least p<0.01).

At the 9^{th} week, the blood glucose level (264.7 mg/dL) of the group fed with the glycine soja powder (DC-p) was lower by 49% than that of the non-administered group (NC). The blood glucose levels of the group fed with the water extract (DC-WE) and the group fed with the methanol extract (DC-ME) were 223.0 mg/dL and 294.6 mg/dL, which were lower by 57% and 43% than the blood glucose level of the NC, respectively. The blood glucose levels of the group fed with the water fraction (DC-WF), the group fed with the ethyl acetate fraction (DC-EF), the group fed with the butanol fraction (DC-BF), and the group fed with the hexane fraction (DC-HF) were 179.4 mg/dL, 253.9 mg/dL, 275.0 mg/dL, and 346 mg/dL, which were lower by 65%, 51%. 47%, and 33% than the blood glucose level of the NC, respectively.

In addition, none of the test groups showed statistically significant differences in body weight, adipose tissue weight, and serum ALT and AST level, demonstrating their non-toxicity.

In conclusion, all of the glycine soja streamed powder, the water and organic solvent extracts of glycine soja, and the solvent fractions of the extracts were effective in the regulation of blood glucose and the amelioration of insulin resistance and lipid metabolism in type 2 diabetes mellitus animal models, in comparison with the control groups. Therefore, intake of the glycine soja streamed powder or the glycine soja extract was evaluated to exhibit superior hypoglycemic efficacy to ameliorate fasting hyperglycemia and efficacy to ameliorate insulin resistance.

During oral administration of the DC60, DC80, DC100, and DC40E extracts, which were prepared by extracting glycine soja at different temperatures, to 4-week-old db/db mouse models for 5 weeks, their hypoglycemic effects were measured [Experimental Example 4]. As a result, the blood glucose level of the group fed with the DC80 extract was 400.5 mg/dL, which was statistically significantly lower (by 37.6%) than that (642.0 mg/ml) of the non-administered group (NC) (p<0.01). In addition, the triglyceride (TG) levels, the total cholesterol levels, the LDL-cholesterol levels, the abdominal fat weights, the liver weights, and the insulin resistance of the groups fed with DC80, DC100, and DC40E were decreased compared to those of the non-administered group.

The efficacies of the glycine soja extracts DC5 and DC25, the column fractions DC60-1 and DC60-2, anthocyanins, pinitol, and banaba/Cr complex (BANABA) were compared and assessed [Experimental Example 5]. Anthocyanins and pinitol are known as antidiabetic active substances derived from plants in the Zingiberaceae family or other natural products. All test samples were found to have hypoglycemic effects at the 9^{th} week and their efficacies decreased in the order: Pinitol (52.1%)>DC25 (47.6%)>DC60-2 (38.6%)>DC5 (34.6%)=BANABA (34.6%)>anthocyanins (26.5%)=DC60-1 (25.8%). At the 9^{th} week, all samples except DC25 and anthocyanins showed suppressive effects on insulin resistance and their efficacies decreased in the order: BANABA (82.4%)>DC60-1 (67.6%)>DC5 (60.9%)=DC60-2 (59.8%)>pinitol (35.3%). Pinitol and BANABA are active ingredients obtained by isolation and purification and were used at high doses or in amounts larger than their general doses. The test groups showed efficacies comparable to the comparative test groups. Taking into consideration the above fact and results, it could be concluded that the test groups had clear hypoglycemic efficacy and suppressive efficacy on insulin resistance.

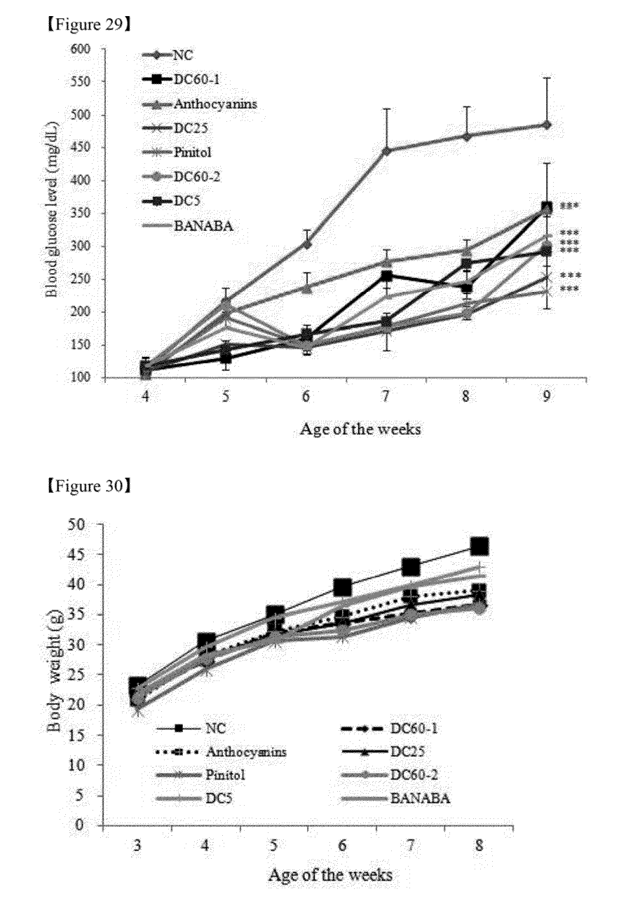

The presence of sequoyitol, chiro-inositol, and pinitol, which are active ingredients of general beans, in the glycine soja extracts and contents thereof were analyzed to confirm whether the antidiabetic effects of the glycine soja extracts were attributed to the active ingredients [Experimental Example 6]. As a result of the analysis, no sequoyitol and chiro-inositol were detected in all of the DC60-1. DC60-2, DC5, and DC25. A slight amount of pinitol was detected, but the hypoglycemic effects of the test materials were not proportional to the pinitol content, which can be seen from the results of Experimental Example 5 (see 2-1 of Experimental Example 5 and FIG. 29). These results show that the active ingredients of the glycine soja streamed powder or extract present in the composition of the present invention are different from the known active ingredients of plants in the Zingiberaceae family.

Glycine soja has been long used as a food material or a crude drug. It is thus apparent that the glycine soja streamed powder and extracts are free from problems, such as toxicity and side effects. This fact was reconfirmed through an acute toxicity experiment in the present invention.

The pharmaceutical composition for preventing and treating diabetes mellitus or diabetic complications according to the present invention includes 0.1 to 99.9% by weight of the glycine soja streamed powder or extract, based on the total weight of the composition.

The pharmaceutical composition of the present invention may further include one or more additives selected from those commonly used in the art, such as carriers, excipients, and diluents.

The glycine soja extract may have any pharmaceutical dosage forms. For example, the glycine soja extract may also be used in the form of a pharmaceutically acceptable salt. The glycine soja extract may be administered alone or in appropriate association as well as in combination with other pharmaceutically active compounds.

The pharmaceutical composition of the present invention may be formulated into preparations for oral application, such as powders, granules, tablets, capsules, suspensions, emulsions, syrups, and aerosols, preparations for external application, suppositories, and sterile injectable preparations. Examples of carriers, excipients and diluents suitable for use in the composition of the present invention include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate and mineral oil. In addition, the pharmaceutical composition of the present invention may be formulated with a diluent or an excipient known in the art, such as a filler, an extender, a binder, a wetting agent, a disintegrant or a surfactant. The pharmaceutical composition of the present invention may be formulated into solid preparations for oral administration. The solid preparations include tablets, pills, powders, granules, and capsules. Such solid preparations are prepared by mixing the glycine soja extract with at least one excipient, for example, starch, calcium carbonate, sucrose, lactose or gelatin. In addition to the excipient, lubricating agents such as magnesium stearate and talc may also be used. The pharmaceutical composition of the present invention may be formulated into liquid preparations for oral administration. The liquid preparations may be suspensions, liquids for internal application, emulsions, and syrups. The liquid preparations may include diluents, for example, water and liquid paraffin. In addition to the diluents, the liquid preparations may include excipients, for example, wetting agents, sweetening agents, flavoring agents, and preservatives. The pharmaceutical composition of the present invention may be formulated into preparations for parenteral administration. The parenteral preparations may include sterile aqueous solutions, non-aqueous solvents, suspensions, emulsions, freeze-dried agents, and suppositories. The non-aqueous solvents and suspensions include propylene glycol, polyethylene glycol, vegetable oils such as olive oil, and injectable esters such as ethyl oleate. As bases for the suppositories, there may be used, for example, Witepsol, Macrogol, Tween 61, cacao butter, laurin butter, and glycerogelatin.

The dosage of the glycine soja extract can be determined by those skilled in the art taking into consideration various factors, such as the health and weight of the patient, severity of disease to be treated, drug form, and route and period of administration. It is recommended to administer the glycine soja steamed powder or extract in an amount ranging from 0.0001 to 10000 g/kg daily. Within this range, the desired effects can be obtained. The daily dose may be administered in a single dose or in divided doses. The dose is in no way intended to limit the scope of the invention.

The glycine soja extract may be administered to mammals such as rats, mice, stock and humans via various routes. All modes of administration can be contemplated. For example, the glycine soja extract may be administered orally, rectally, or by intravenous, intramuscular, subcutaneous, epidural or intracerebroventricular injection.

Definitions for the terms “excipient”, “binder”, “disintegrant”, “lubricating agent”, “flavoring agent”, and “flavor” used herein are described in the literature and are intended to include those that are functionally identical or similar (Explanation Section of the Korean Pharmacopoeia, Munseongsa, The Korean Association of Colleges of Pharmacy, 5^{th} edition, pp. 33-48, 1989).

The food composition for preventing and ameliorating diabetes mellitus or diabetic complications according to the present invention includes 0.1 to 99.9% by weight of the glycine soja streamed powder or extract, based on the total weight of the composition. The food is intended to include health functional foods. The term “health functional foods” used herein means foods that are produced and processed from raw materials or ingredients having functionalities good for human health. The term “functionalities” mean that nutrients are regulated to strengthen the structure and functions of the human body and are ingested to obtain health effects such as useful physiological functions. The health functional foods may have formulations selected from tablets, capsules, powders, granules, liquids, and pills.

The food composition of the present invention may be prepared by adding the glycine soja streamed powder or extract to a food or beverage for the purpose of ameliorating blood glucose and lipid metabolism and effectively preventing and treating diabetic complications. Particularly, the food composition of the present invention may be provided in the form of a health supplement food. For example, the food may be selected from beverages, powdered beverages, solid foods, chewing gums, teas, vitamin complexes, and food additives.

The food composition of the present invention may further include one or more ingredients, in addition to the glycine soja streamed powder or extract as an essential ingredient. There is no particular restriction on the kind of the additional ingredients. For example, the additional ingredients may be those that are commonly used in foods and beverages, for example, flavoring agents and natural carbohydrates. Examples of preferred natural carbohydrates include: monosaccharide such as glucose and fructose; disaccharides such as maltose and sucrose: polysaccharides such as dextrin and cyclodextrin; and sugar alcohols such as xylitol and erythritol. Examples of preferred flavoring agents include: natural flavoring agents such as taumatin, stevia extract (e.g., levaudioside A and glycyrrhizin); and synthetic flavoring agents such as saccharin and aspartame. The natural carbohydrates may be generally used in an amount of about 1 to about 20 g, preferably about 5 to about 12 g per 100 ml of the composition.

The composition of the present invention may further contain various nutritional supplements, vitamins, minerals (electrolytes), synthetic and natural flavoring agents, coloring agents, and fillers (e.g., cheese and chocolate), pectic acid and salts thereof, alginic acid and salts thereof, organic acids, protective colloid thickeners, pH-adjusting agents, stabilizers, preservatives, glycerin, alcohols, carbonating agents used in carbonated drinks, etc. The composition of the present invention may further contain fruit pulps for the production of natural fruit juices, fruit juice beverages, and vegetable beverages. These ingredients may be used independently or in combination of two or more thereof. The ratio of such additives is not critical and is generally in the range of 0 to about 20% by weight, based on the total weight of the composition.

The present invention will be explained in detail with reference to the following examples, including experimental examples. However, these examples are provided for illustrative purposes only and are not intended to limit the scope of the invention.

### Example 1

### Preparation of Glycine soja Streamed Powder

1. Glycine soja Powder

Glycine soja growing naturally in the region of Uji-dong, Mungyeong-si, Kyeongsangbuk-do, Korea was harvested and dried. The dried glycine soja was ground using a blender to obtain a glycine soja powder.

2. Glycine soja Streamed Powder

The glycine soja powder obtained in 1 was steamed at about 80° C. for about 2 h to obtain a heat-treated powder of glycine soja (hereinafter referred to as “DC”).

### Example 2

### Preparation of Glycine soja Extracts and Fractions

1. Glycine soja Water Extract

10 L of water was added to 1 kg of the glycine soja powder prepared in 1 of Example 1. The glycine soja powder was extracted at 60° C. for 4 h. The extract solution was filtered through a filter paper (Whatman, USA) under reduced pressure. Thereafter, the filtrate was collected and concentrated using a rotary evaporator at 45° C. to obtain 130 g of a water extract of the glycine soja powder (hereinafter referred to as “DC-WE”).

2. Glycine soja Methanol Extract

10 L of methanol was added to 1 kg of the glycine soja powder prepared in 1 of Example 1. The glycine soja powder was extracted at 60° C. for 4 h. The extract solution was filtered through a filter paper (Whatman, USA) under reduced pressure. Thereafter, the filtrate was collected and concentrated using a rotary evaporator at 37° C. to obtain 180 g of a methanol extract of the glycine soja powder (hereinafter referred to as “DC-ME”)

3. Glycine soja Solvent Fractions

100 g of the glycine soja methanol extract prepared in 2 was suspended in 2 L of distilled water, and the same amount of hexane was added thereto. After shaking, the mixture was fractionated twice for layer separation. To the water layer was added the same amount of ethyl acetate. After shaking, the mixture was fractionated twice for layer separation. To the water layer was added the same amount of water-saturated butanol. After shaking, the mixture was fractionated twice for layer separation. The obtained organic solvent fractions were dried under reduced pressure and the water fraction was freeze-dried to obtain 19.6 g of a hexane fraction (hereinafter referred to as “DC-HF”), 12.7 g of an ethyl acetate fraction (hereinafter referred to as “DC-EF”), 29.0 g of a butanol fraction (hereinafter referred to as “DC-BF”), and 27.7 g of a water fraction (hereinafter referred to as “DC-WF”).

### Example 3

### Preparation of Glycine soja Extracts at Different Extraction Temperatures

10 L of water was added to 1 kg of the glycine soja powder prepared in 1 of Example 1 and different portions of the mixture were extracted with water at 60° C., 80° C., and 100° C. for 4 h. Each extract solution was filtered through a filter paper (Whatman, USA) under reduced pressure. Thereafter, the filtrate was collected and concentrated using a rotary evaporator at 45° C. to obtain a water extract. The water extracts obtained at the different temperatures of 60° C., 80° C. and 100° C. are referred to as “DC60”, DC80, and DC100, respectively. An ethanol extract was prepared in the same manner as above, except that ethanol was used instead of water and extraction was performed at 40° C. This ethanol extract is referred to as “DC40E.”

### Experimental Example 1

### Effects of the Glycine soja Streamed Powder on Diabetes Mellitus and Diabetic Complications

1. Materials and Methods

1-1. Test Materials and Administration Methods

The glycine soja streamed powder obtained in 2 of Example 1 was diluted with physiological saline. The dilution was administered orally twice a day (10 a.m. and 6 p.m.). A banaba leaf extract in the form of a brown tablet (Wellness banaba Co. Ltd.) was pulverized into a powder, which was then dissolved in physiological saline. The solution was used as positive control. The positive control was administered orally twice a day (10 a.m. and 6 p.m.). The same amount of physiological saline as negative control was administered in the same manner.

1-2. Administration of Antidiabetic Agents, Efficacy Evaluation, and Preliminary Animal Experiment

6-week-old male db/db mice (each 26 g, Taconic Farms, Inc.) were divided into 3 groups, 7 mice per group. The antidiabetic agent was administered to each group. The animals were maintained in a specific pathogen free (SPF) breeding environment at a temperature of 22±2° C. and a humidity of 55±10% on a 12 h light-12 h dark cycle. Immediately after and the 3^{rd}, 6^{th}, 9^{th}, 12^{th}, and 15^{th} days after sample administration, the body weights and blood glucose levels of the animals were measured to determine an optimum concentration at which the hypoglycemic efficacy of each sample was confirmed in the animals.

1-3. Administration of Antidiabetic Agents, Efficacy Evaluation, Animal Experiment and Sampling

Obese 6-week-old male db/db mice with diabetic symptoms are experimental animals in which genetic variation is artificially induced to block feedback signal transmission from leptin, and a result, type 2 diabetes mellitus is induced with excess weight gain during growth. The samples were administered orally to the mouse models. The mouse models were randomly divided into 3 groups, 7 db/db mice per group. One of the groups was a test group and 2 g/kg of the glycine soja streamed powder (DC) was administered orally at 10 a.m. and 6 p.m. daily. 100 mg/kg of the banaba leaf extract as positive control was administered in the same manner as the test group. The same amount of physiological saline as negative control was administered in the same manner as the test group. The animals were maintained in an SPF environment at a temperature of 22±2° C. and a humidity of 55±10% on a 12 h light-12 h dark cycle. Immediately after and the 7^{rd}, 14^{th}, and 21^{st} days after administration, changes in the body weight and blood glucose level of the animals were measured using a portable blood glucose meter (OneTouch™, Johnson & Johnson, USA). The values in each experimental group were averaged.

1-4. Hemanalysis, Autopsy, and Histopathological Analysis

Blood was drawn from each experimental animal and centrifuged at 3,000 rpm for 10 min. The supernatant plasma was separated and analyzed for glucose and triglyceride. For blood biochemical indices associated with blood glycolipid and other organ toxicity, significant differences between the test group and the control groups were analyzed using Student's T-test.

For histopathological observation of organs, liver, kidney, heart, pancreas, and lung were excised and stored in 10% neutral buffered formalin, and their portions were stored in RNASol^{B}.

1-5. Statistical Processing

Data obtained from the above experiments were expressed as mean±standard error and significance assay was determined using Student's T-test.

2. Results

2-1. Blood Glucose Changes

The test group (DC, 2 g/kg), the positive control (banaba leaf extract 100 mg/kg), and negative control (physiological saline) were administered orally to 6-week-old db/db mouse models twice (in the morning and afternoon) a day for 3 weeks. Blood samples were drawn from the mouse tail vein and blood glucose levels were measured using a blood glucose meter once a week. All mice were fasted (for about 12 h) on the day before measurement. As a result of the experiment, blood glucose changes are shown in FIG. 1. As shown in FIG. 1, the blood glucose level of the negative control (NC) increased to 582.1 mg/dL at the age of 9 weeks, which was higher by 70% or more than that (342.1 mg/dL) at the age of 6 weeks. The mice experienced a 70% or more increase in average blood glucose level for the 3 weeks. It was found that the blood glucose level of the DC-administered group was considerably decreased compared to that of the NC. The blood glucose level (360.5 mg/kg) of the DC-administered group was 360.5 mg/kg, which corresponds to a 37.9% (˜40%) decrease compared to that of the NC (p<0.001). The blood glucose of the DC-administered group was drastically decreased even compared to that of the group fed with the banaba leaf extract as positive control.

2-2. Serum Glucose Changes

The experiment was completed at the age of 9 weeks. All mice were fasted (for about 12 h) on the day before measurement and anesthetized with ethyl ether. Blood was drawn from the heart of each animal with a 3 ml syringe and left standing at room temperature for 1 h. Thereafter, the blood was centrifuged at 3000 rpm for 10 min to separate serum. Changes in serum glucose level were measured using an automatic serum analyzer. The results are shown in FIG. 2. The glucose level of the NC was 641 mg/dL and that of the test group fed with the glycine soja streamed powder (DC) was 427 mg/dL. The DC-administered group showed a significant decrease (33.4%) in serum glucose level compared with the NC (p<0.01). The glucose level (463 mg/dL) of the banaba leaf extract as positive control was statistically significantly decreased (27.7%) compared with that of the NC (p<0.01), but the test group (DC) had superior hypoglycemic effects to the positive control.

2-3. Effects on Lipid Metabolism and Diabetic Complications

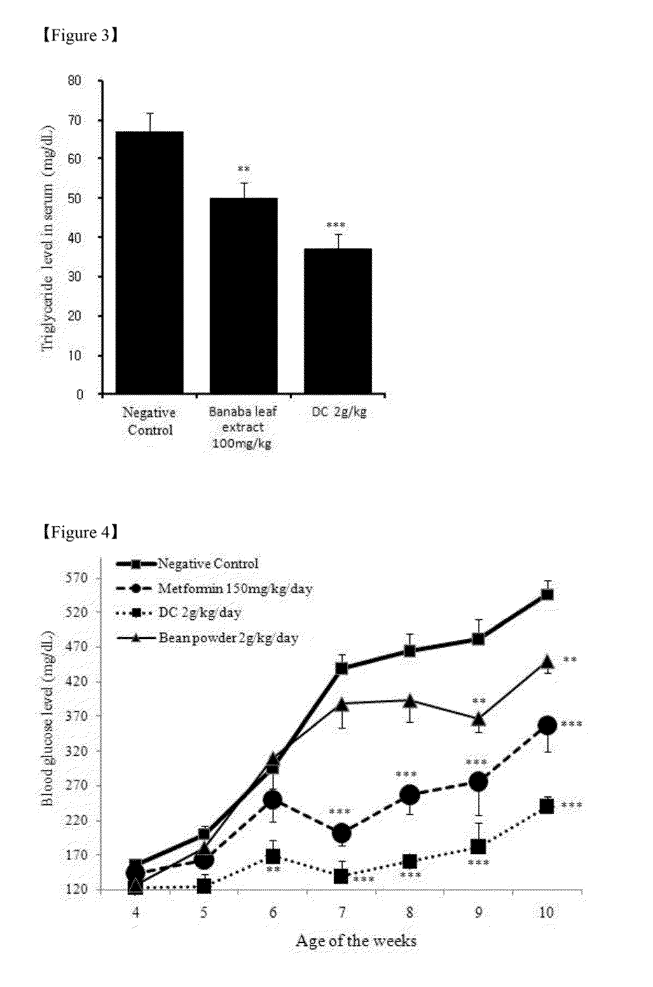

Serum triglyceride (TG) level changes were measured to confirm effects on lipid metabolism and diabetic complications. The experiment was completed at the age of 9 weeks. All mice were fasted (for about 12 h) on the day before measurement and anesthetized with ethyl ether. Blood was drawn from the heart of each animal with a 3 ml syringe and left standing at room temperature for 1 h. Thereafter, the blood was centrifuged at 3000 rpm for 10 min to separate serum. Changes in serum triglyceride level were measured using an automatic serum analyzer. The results are shown in FIG. 3. As can be seen from FIG. 3, the triglyceride (TG) level of the NC was 67.0 mg/dL and that of the test group fed with the glycine soja streamed powder (DC) was 37.01 mg/dL. The DC-administered group showed a statistically significant decrease (44.7% or more) in serum triglyceride level compared with the NC (p<0.001). These results are in substantial agreement with the suppressive efficacy results on serum glucose obtained in 2-2, demonstrating that the DC was effective in improving blood circulation. The triglyceride level (50 mg/dL) of the banaba leaf extract as positive control was statistically significantly decreased (25.3%) compared with that of the NC (p<0.01), but the DC-administered group was very effective in decreasing triglyceride level compared to the positive control.

2-4. Acute Toxicity Experiment

An acute toxicity experiment was conducted using specific pathogen-free (SPF) SD rats, aged 6 weeks. 5 rats were assigned per group. 5,000 mg/kg of the glycine soja streamed powder (DC) was orally administered once to each animal. After administration, life or death, clinical symptoms and body weight changes of the animals were observed and hematological and blood biochemical examinations were conducted. After autopsy, a visual observation was made as to whether abnormalities occurred in the abdominal and thoracic organs. As a result of the experiment, no noticeable clinical symptoms were observed in all animals fed with the test material and none of the animals died. No toxicity changes were observed even in the body weight change, blood examination, blood biochemical examination and autopsy report. The foregoing results showed that the glycine soja streamed powder (DC) caused no toxicity changes in the rats even in an amount of 5,000 mg/kg and had a lethal dose 50% (LD₅₀) of 5,000 mg/kg or more upon oral administration, demonstrating safety of the DC.

### Experimental Example 2

### Comparison of Antidiabetic Efficacies of the Glycine soja Streamed Powder and Metformin

1. Materials and Methods

1-1. Test Materials and Administration Methods

The glycine soja streamed powder (DC) was diluted with physiological saline and the dilution was orally administered in divided portions (each 0.2 ml) at 10 a.m. and 4 p.m. daily using a mouse sonde (2 g/kg/day). Metformin (1,1-dimethylbiguanide), which is a widely used hypoglycemic agent, was chosen as positive control. Metformin was dissolved in 0.25% carboxymethylcellulose (CMC) and administered orally in two divided portions (each 0.2 ml) in the same manner (150 mg/kg/day). The same amount of physiological saline as negative control was administered in the same manner. A general bean streamed powder was used as another comparative test group. The bean streamed powder was obtained in the same manner as in Example 1, except that bean was used instead of glycine soja. The bean streamed powder was administered orally twice a day in the same amount and manner as the glycine soja streamed powder (DC).

1-2. Experimental Group Classification and Experimental Design

Male db/db mice, aged 3 weeks, were acclimatized to an animal breeding room for 1 week. 6 mice were assigned to each experimental group. The blood glucose levels of the animals were measured from the age of 4 weeks at intervals of 1 week for a total of 6 weeks. Drug was administered orally in divided portions (each 0.2 ml) at 10 a.m. and 4 p.m. daily using a mouse sonde. All animals were fasted for 6 h from 9 a.m. to 3 p.m. every Wednesday. Blood samples were collected from the mouse tail vein and blood glucose levels were measured using a portable blood glucose meter (OneTOUCH@Ultra. Johnson & Johnson, USA). Since serum for final biochemical analysis requires blood sampling after fasting for at least 12 h, blood glucose levels were measured at the final 8^{th} week. 2 days after the measurement, an autopsy was conducted. For the autopsy, all animals were fasted for 12 h. Blood samples were drawn from the heart under ether anesthesia, put in tubes for serum separation, and centrifuged at 3000 rpm for 20 min to obtain serum samples, which were used as samples for the analysis of biochemical indices.

Experimental Group Classification
Control (db/db mice) (n=6)
Positive control (Metformin, 150 mg/kg/day, o.p) (n=6)
Glycine soja streamed powder (DC) (2.0 g/kg/day, o.p) (n=6)
Bean powder (2.0 g/kg/day, o.p) (n=6)

1-3. Blood Glucose and Insulin Analysis

After a test material was administered to each experimental group for 6 weeks, blood glucose level was measured at the final 8^{th} week. 2 days after the measurement, an autopsy was conducted. For the autopsy, each animal was fasted for 12 h. Blood was drawn from the heart under ether anesthesia, put in a tube for serum separation, and centrifuged at 3000 rpm for 20 min to obtain serum, which was used as a sample for the analysis of biochemical indices. The glucose and insulin levels of the serum were analyzed. Changes in the insulin level and insulin resistance index (HOMAIR) of each experimental group during administration for 8 weeks period were calculated by the following formulae. Plasma was obtained as a sample in the same manner. The level of insulin in the plasma was measured using a Mouse Insulin ELISA kit (Shibayagi, Japan) and an ELISA reader (Labsystems, Finland).

1-4. Measurement of Adipose Tissue Weight

After a test material was administered to each experimental group for 6 weeks, abdominal, epididymal, and inguinal adipose tissues were excised from each experimental animal. The total weight of the adipose tissues was measured.

1-5. Biochemical Analysis of Plasma Lipids

Blood was obtained in the same manner as in 1-3, put in a tube for serum separation, and centrifuged at 3000 rpm for 20 min to obtain serum, which was used as a sample for the analysis of biochemical indices. Total cholesterol, high-density lipoprotein (HDL) cholesterol, low-density lipoprotein (LDL) cholesterol, triglyceride, and phospholipid levels, which are indices of lipid contents in plasma and liver, in the plasma were measured using an automatic biochemical analyzer (Hitachi-720, Hitachi Medical, Japan).

1-6. Statistical Processing

Data obtained from the above experiments were expressed as mean±standard error and significance assay was determined using Student's T-test.

2. Results

2-1. Blood Glucose Changes

Metformin as positive control (150 mg/kg/day), the glycine soja streamed powder (DC) as test group (2 g/kg/day), and the bean powder as comparative test group (2 g/kg/day) were administered orally to 4-week-old db/db mouse models twice (in the morning and afternoon) a day for 6 weeks. Blood samples were drawn from the mouse tail vein and blood glucose levels were measured using a blood glucose meter once a week. All mice were fasted 6 h before measurement. The experiment results are shown in FIG. 4. As shown in FIG. 4, the blood glucose level of the negative control (NC) increased from 155.8 mg/dL at the age of 4 weeks to 545.8 mg/dL at the age of 6 weeks, indicating a 3.5-fold increase for 6 weeks. The blood glucose level of the group fed with the glycine soja streamed powder (DC) was 240.5 mg/dL at the age of 10 weeks, which was statistically significantly lower (by 55.9% or more) than that (545.8 mg/dL) of the non-administered group (NC) (p<0.001). The blood glucose levels of the group fed with metformin as positive control and the group fed with the bean powder as comparative test group were statistically significantly lower (by 34.6% and 17.6%, respectively) than the blood glucose level of the non-administered group (NC) (p<0.001, p<0.01). The blood glucose levels of the group fed with the glycine soja streamed powder (DC) and the group fed with metformin as positive control were greatly suppressed compared to the blood glucose of the non-administered group (NC) for 6 weeks. Particularly, the DC-administered group showed much better suppressive effects on blood glucose than the metformin-administered group.

2-2. Serum Glucose Changes

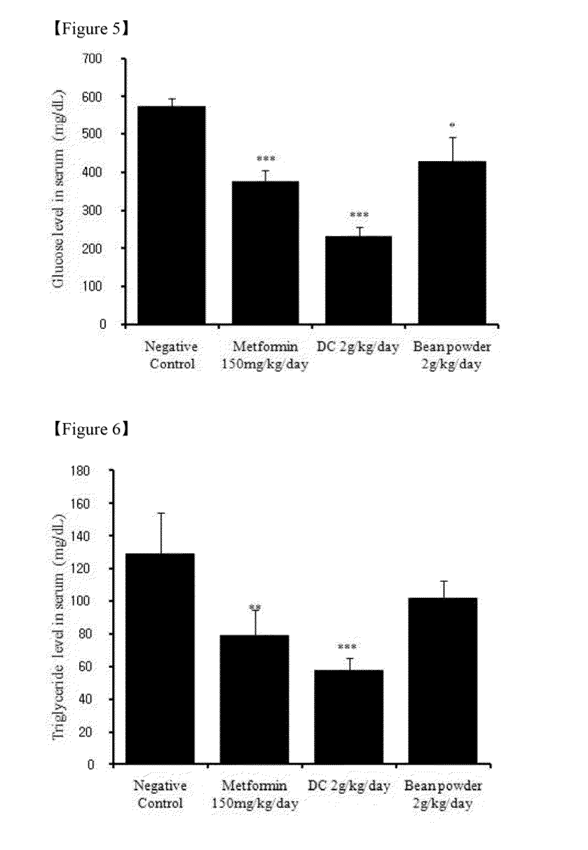

The experiment was completed at the age of 10 weeks. All mice were fasted (for about 16 h) on the day before measurement and anesthetized with ethyl ether. Blood was drawn from the heart of each animal with a 3 ml syringe and left standing at room temperature for 1 h. Thereafter, the blood was centrifuged at 3000 rpm for 10 min to separate serum. Changes in serum glucose level were measured using an automatic serum analyzer. The results are shown in FIG. 5. The serum glucose level of the non-administered group (NC) was 575.0 mg/dL and the serum glucose levels of the group fed with metformin as positive control and the test group fed with the glycine soja streamed powder (DC) were 376.0 mg/dL and 231.0 mg/dL, which were statistically significantly lower (by 34.6% and 59.8%, respectively) than the serum glucose level of the non-administered group (p<0.001). Particularly, the DC-administered group showed a considerable decrease in serum glucose level compared to the group fed with metformin as positive control. These results are in substantial agreement with the results of FIG. 4 showing changes in the glucose level in the blood from the tail vein and are consistent with real-time antidiabetic effects according to the DC administration. The serum glucose level of the group fed with the bean powder as comparative test group was 426.3 mg/dL, which was significantly lower (by 25.8%) than that of the non-administered group (p<0.05), but the decrement in the serum glucose level of the group fed with the bean powder was much lower than that of the DC-administered group.

2-3. Serum Triglyceride (TG) Changes

The experiment was completed at the age of 10 weeks. All mice were fasted (for about 16 h) on the day before measurement and anesthetized with ethyl ether. Blood was drawn from the heart of each animal with a 3 ml syringe and left standing at room temperature for 1 h. Thereafter, the blood was centrifuged at 3000 rpm for 10 min to separate serum. Changes in serum triglyceride level were measured using an automatic serum analyzer. The results are shown in FIG. 6. The serum triglyceride levels of the groups fed with metformin as positive control and DC were 78.8 mg/dL and 57.2 mg/dL, which were statistically significantly lower (by 38.8% and 55.6%, respectively) than the serum triglyceride level (128.8 mg/dL) of the non-administered group (NC) (p<0.01, p<0.001). Particularly, the DC-administered group showed a considerable decrease in serum triglyceride level compared to the group fed with metformin as positive control. These results suggest that the DC is more effective in improving blood circulation than metformin as positive control due to its better ability to reduce triglyceride level. However, the triglyceride (TG) level of the group fed with the bean powder was 101.8 mg/dL, which was not significantly different from that of the non-administered group.

2-3. Serum Total Cholesterol Changes

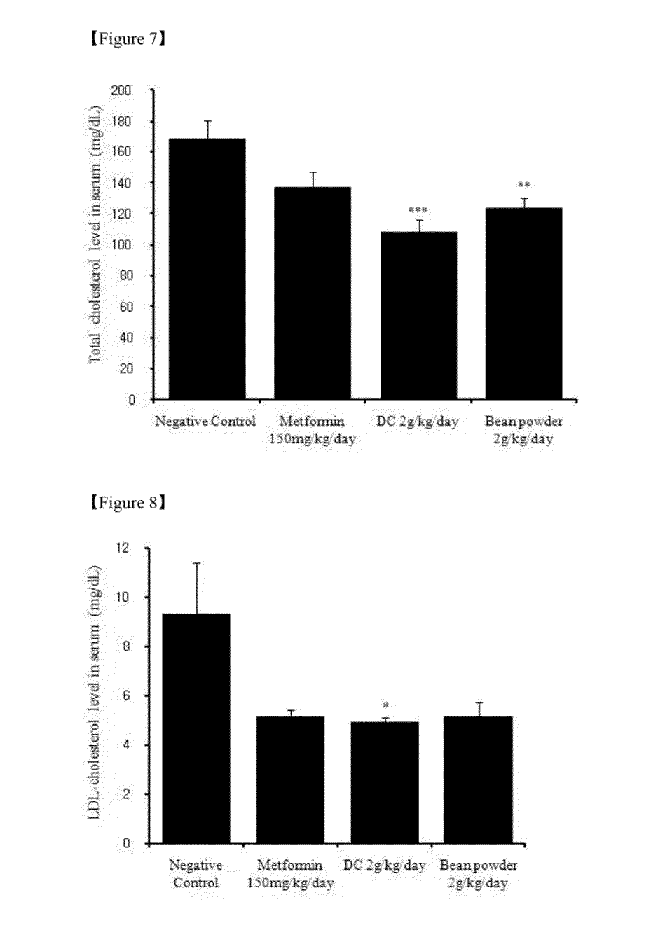

The experiment was completed at the age of 10 weeks. All mice were fasted (for about 16 h) on the day before measurement and anesthetized with ethyl ether. Blood was drawn from the heart of each animal with a 3 ml syringe and left standing at room temperature for 1 h. Thereafter, the blood was centrifuged at 3000 rpm for 10 min to separate serum. The level of total cholesterol in the serum was measured using an automatic serum analyzer. The results are shown in FIG. 7. The serum total cholesterol level of the non-administered group (NC) was 168.2 mg/dL and that of the DC-administered group was 107.7 mg/dL, which was statistically significantly lower (by 35.9%) than that of the NC (p<0.001).

The total cholesterol level of the metformin-administered group was low compared to that of the non-administered group, but there was no statistical significance between the two groups. The total cholesterol level of the group fed with the bean powder was 123.2 mg/dL, which was significantly lower (by 26.7%) than that of the non-administered group (p<0.01). These results indicated that the DC administration was most effective in decreasing total cholesterol level, leading to improved blood circulation.

2-4. Serum Low-Density Lipoprotein Cholesterol Changes

The experiment was completed at the age of 10 weeks. All mice were fasted (for about 16 h) on the day before measurement and anesthetized with ethyl ether. Blood was drawn from the heart of each animal with a 3 ml syringe and left standing at room temperature for 1 h. Thereafter, the blood was centrifuged at 3000 rpm for 10 min to separate serum. The level of low-density lipoprotein cholesterol in the serum was measured using an automatic serum analyzer. The results are shown in FIG. 8. The serum low-density lipoprotein cholesterol level of the non-administered group (NC) was 9.3 mg/dL and that of the DC-administered group was 4.9 mg/dL, which was statistically significantly lower (by 47.3%) than that of the NC (p<0.05). The low-density lipoprotein cholesterol levels of the metformin-administered group and the bean powder-administered group were low compared to the low-density lipoprotein cholesterol level of the non-administered group, but there were no statistical significances among the groups. These results indicated that the DC administration was most effective in decreasing low-density lipoprotein cholesterol level, leading to improved blood circulation.

2-5. Serum High-Density Lipoprotein Cholesterol Changes

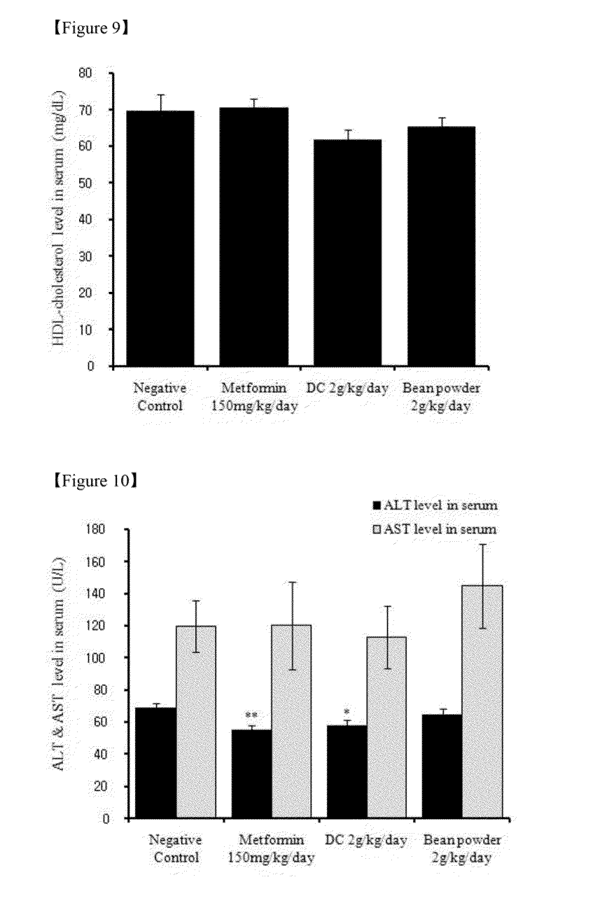

The experiment was completed at the age of 10 weeks. All mice were fasted (for about 16 h) on the day before measurement and anesthetized with ethyl ether. Blood was drawn from the heart of each animal with a 3 ml syringe and left standing at room temperature for 1 h. Thereafter, the blood was centrifuged at 3000 rpm for 10 min to separate serum. The level of high-density lipoprotein cholesterol in the serum was measured using an automatic serum analyzer. The results are shown in FIG. 9. The metformin-administered group, the DC-administered group, and the bean powder-administered group showed no significant differences in high-density lipoprotein cholesterol level compared with the non-administered group (NC).

2-6. Serum ALT and AST Changes

The experiment was completed at the age of 10 weeks. All mice were fasted (for about 16 h) on the day before measurement and anesthetized with ethyl ether. Blood was drawn from the heart of each animal with a 3 ml syringe and left standing at room temperature for 1 h. Thereafter, the blood was centrifuged at 3000 rpm for 10 min to separate serum. The levels of ALT and AST in the serum were measured using an automatic serum analyzer. The results are shown in FIG. 10. The ALT levels of the non-administered group (NC), the group fed with metformin as positive control, the DC-administered group, and the bean powder-administered group were 68.5 U/L, 54.9 U/L, 57.4 U/L, and 64.6 U/L, respectively, and no hepatotoxicity was observed in all experimental groups. The AST levels of the non-administered group (NC), the metformin-administered group, the DC-administered group, and the bean powder-administered group were 119.7 U/L, 120.0 U/L, 112.7 U/IL, and 144.5 U/L, respectively, and no hepatotoxicity was observed in all experimental groups.

2-7. Serum Insulin Changes

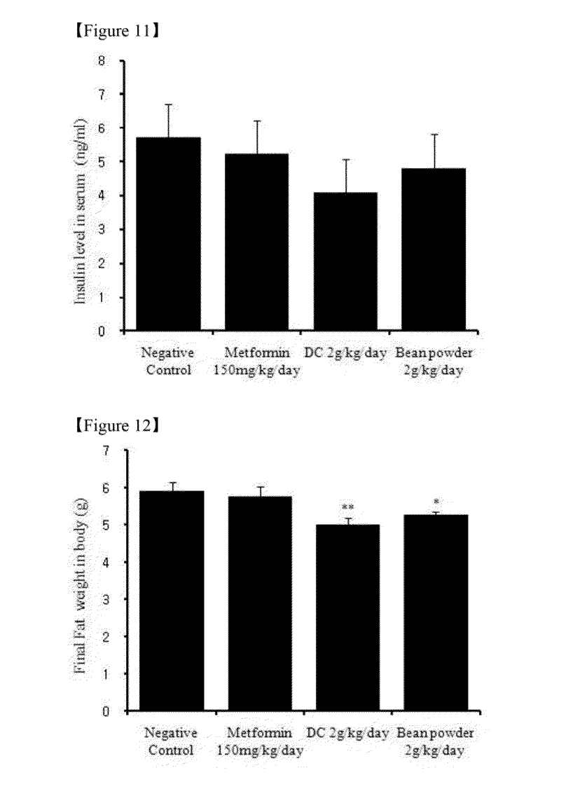

The experiment was completed at the age of 10 weeks. All mice were fasted (for about 16 h) on the day before measurement and anesthetized with ethyl ether. Blood was drawn from the heart of each animal with a 3 ml syringe and left standing at room temperature for 1 h. Thereafter, the blood was centrifuged at 3000 rpm for 10 min to separate serum. The level of insulin in the serum was measured using a Mouse Insulin ELISA kit (SHIBAYAGI, Japan). The results are shown in FIG. 11. The serum insulin level of the non-administered group (NC) was 5.72 ng/ml and that of the DC-administered group was 4.07 mg/dL, which was lower by 28.8% than that of the NC (p<0.001), but there was no statistical significance between the two groups. The serum insulin levels of the metformin-administered group and the general bean-administered group were slightly lower than the serum insulin level of the non-administered group, but there were no statistical significances among the groups. It is known that patients with type 2 diabetes mellitus normally secrete insulin but possess insulin resistance due to increased resistance of blood glucose to insulin, and as a result, the level of insulin in the blood of the patients increases, causing various metabolic disorders. The results of this experiment show that the DC has suppressive efficacy on blood glucose and effectively ameliorates resistance to insulin, which is a problem of type 2 diabetes mellitus.

2-8. Adipose Tissue Changes

After the experiment was completed at the age of 10 weeks, abdominal, epididymal, and inguinal adipose tissues were excised from each db/db mouse. The total weight of the adipose tissues was measured. The results are shown in FIG. 12. The adipose tissue weight of the non-administered group (NC) was 5.9 g and that of the DC-administered group was 5.0 g, which was lower by 15.3% than that of the NC, but there was no statistical significance between the two groups (p<0.01).

### Experimental Example 3

### Effects of the Glycine soja Extracts and Solvent Fractions Diabetes Mellitus and Diabetic Complications

1. Materials and Methods

1-1. Diabetes Mellitus Model Animals and Experimental Design

Male db/db mice, aged 3 weeks, were acclimatized to an animal breeding room for 1 week. 6 mice were assigned to each experimental group. Drug was administered orally in divided portions (each 0.2 ml) at 10 a.m. and 4 p.m. daily using a mouse sonde. The blood glucose levels of the animals were measured from the age of 4 weeks at intervals of 1 week for a total of 6 weeks. All animals were fasted for 6 h from 9 a.m. to 3 p.m. every Wednesday. Blood samples were collected from the mouse tail vein and blood glucose levels were measured using a portable blood glucose meter (OneTOUCH@Ultra, Johnson & Johnson, USA). The overall experimental design was the same as that in Experimental Example 2.

1-2. Test Materials and Administration Methods

The glycine soja streamed powder obtained in Example 1, and the glycine soja extracts and fractions obtained in Example 2 were used as test materials. Each of the test materials was suspended in physiological saline and administered orally in divided portions (each 0.2 ml) at 10 a.m. and 4 p.m. daily using a mouse sonde. The following are a total of 8 experimental groups used.

Experimental Groups:
Control group (db/db mice) (n=6)
Glycine soja water extract (DC-WE) (300 mg/kg, o.p) (n=6)
Glycine soja methanol extract (DC-ME) (300) mg/kg, o.p) (n=6)
Hexane solvent fraction of glycine soja methanol extract (DC-HF) (100 mg/kg, o.p) (n=6)
Butanol solvent fraction of glycine soja methanol extract (DC-BF) (100 mg/kg, o.p) (n=6)
Ethyl acetate solvent fraction of glycine soja methanol extract (DC-EF) (100 mg/kg, o.p) (n=6)
Water solvent fraction of glycine soja methanol extract (DC-WF) (100 mg/kg, o.p) (n=6)
Glycine soja streamed powder (DC-p) (1.5 g/kg, o.p) (n=6)

1-3. Measurement of Adipose Tissue Weights

After administration of each test material for 6 weeks, abdominal, epididymal, and inguinal adipose tissues were excised from each experimental animal. The total weight of the adipose tissues was measured.

1-4. Biochemical Analysis of Plasma Lipids of db/db Mice with Type 2 Diabetes Mellitus

After each test material was administered for 6 weeks, blood glucose level was measured at the final 8^{th} week. 2 days after the measurement, an autopsy was conducted. For the autopsy, each animal was fasted for 12 h. Blood was drawn from the heart under ether anesthesia, put in a tube for serum separation, and centrifuged at 3000 rpm for 20 min to obtain serum, which was used as a sample for the analysis of biochemical indices. After plasma was separated. ALT and AST levels, which are liver function indices, and total cholesterol, high-density lipoprotein (HDL) cholesterol, low-density lipoprotein (LDL) cholesterol, and triglyceride levels, which are indices of lipid contents in plasma and liver, in the plasma were measured using an automatic biochemical analyzer (Hitachi-720. Hitachi Medical, Japan).

1-5, Serum Glucose and Insulin Analysis

Serum was separated in the same manner as in 1-4. The levels of glucose and insulin in the serum were analyzed. Plasma was obtained as a sample in the same manner. The level of insulin in the plasma was measured using a Mouse Insulin ELISA kit (Shibayagi, Japan) and an ELISA reader (Labsystems, Finland).

1-6. Statistical Processing

Data obtained from the above experiments were expressed as mean±standard error and significance assay was determined using Student's T-test.

2. Results

2-1. Blood Glucose Changes

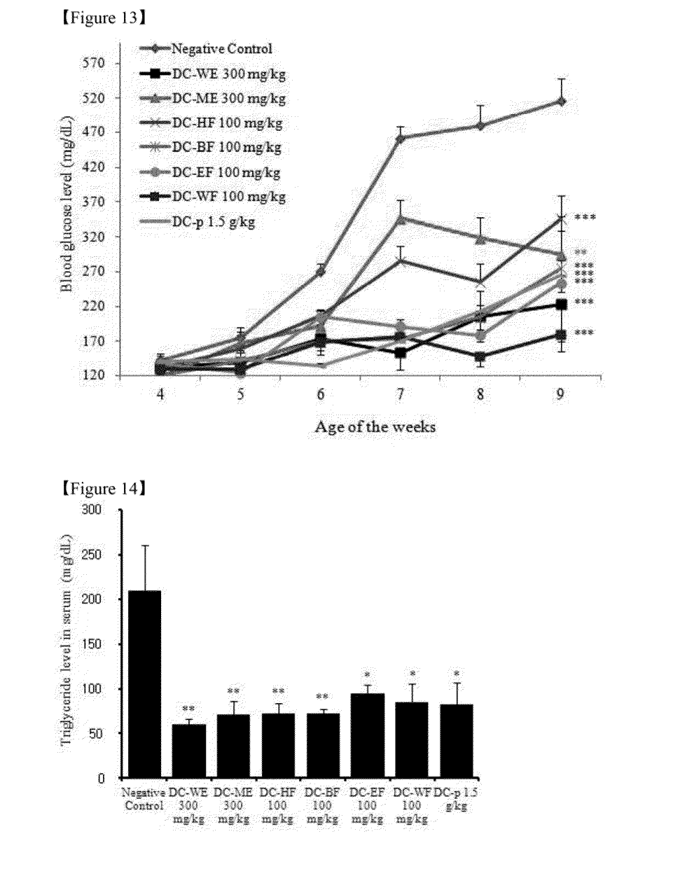

The glycine soja water extract (300 mg/kg/day, DC-WE), the methanol extract (300 mg/kg, DC-ME), the hexane fraction (100 mg/kg/day, DC-HF), the BuOH fraction (100 mg/kg/day, DC-BF), the ethyl acetate (EtOAC) fraction (100 mg/kg/day, DC-EF), the water fraction (100 mg/kg/day, DC-WF), and the glycine soja streamed powder (DC-p, 1.5 g/kg/day, DC-p) were administered orally to 4-week-old db/db mouse models twice (in the morning and afternoon) a day for 5 weeks. Blood samples were drawn from the mouse tail vein and blood glucose levels were measured using a portable blood glucose meter once a week. All mice were fasted from 6 h before measurement. The results are shown in FIG. 13. The blood glucose level of the negative control (NC) steadily increased to 142.8 mg/dL at the age of 4 weeks, 175.8 mg/dL at the age of 5 weeks, 270.5 mg/dL at the age of 6 weeks, 461.5 mg/dL at the age of 7 weeks, and 479.8 mg/dL at the age of 8 weeks. The blood glucose level of the negative control at the final 9^{th} week was 516.3 mg/dL, which was 3.6 times higher than that at the age of 4 weeks. In contrast, the blood glucose levels of all test groups of glycine soja were statistically significantly lower than those of the NC (at least p<0.01).

At the 9^{th} week, the blood glucose level (264.7 mg/dL) of the group fed with the glycine soja streamed powder (DC-p) was lower by 49% than that of the NC. The blood glucose levels of the group fed with the water extract (DC-WE) and the group fed with the methanol extract (DC-ME) were 223.0 mg/dL and 294.6 mg/dL, which were lower by 57% and 43% than the blood glucose level of the NC, respectively. The blood glucose levels of the group fed with the water fraction (DC-WF), the group fed with the ethyl acetate fraction (DC-EF), the group fed with the butanol fraction (DC-BF), and the group fed with the hexane fraction (DC-HF) were 179.4 mg/dL, 253.9 mg/dL, 275.0 mg/dL, and 346 mg/dL, which were lower by 65%, 51%, 47%, and 33% than the blood glucose level of the NC, respectively.

2-2. Serum Triglyceride (TG) Changes

The experiment was completed at the age of 9 weeks. All mice were fasted (for about 16 h) on the day before measurement and anesthetized with ethyl ether. Blood was drawn from the heart of each animal with a 3 ml syringe and left standing at room temperature for 1 h. Thereafter, the blood was centrifuged at 3000 rpm for 10 min to separate serum. Changes in serum triglyceride (TG) level were measured using an automatic serum analyzer. The results are shown in FIG. 14. The serum triglyceride (TG) level of the non-administered group was 209.3 mg/dL and the serum glucose levels of the groups fed with DC-WE, DC-ME, DC-HF, DC-BF, DC-EF, DC-WF, and DC-p were statistically significantly lower (by at least 54.0%) than the serum glucose level of the non-administered group (p<0.05, p<0.01). These results confirmed that the glycine soja streamed powder, the glycine soja extracts, and the glycine soja fractions were remarkably effective in improving blood circulation.

2-3. Serum Total Cholesterol Changes

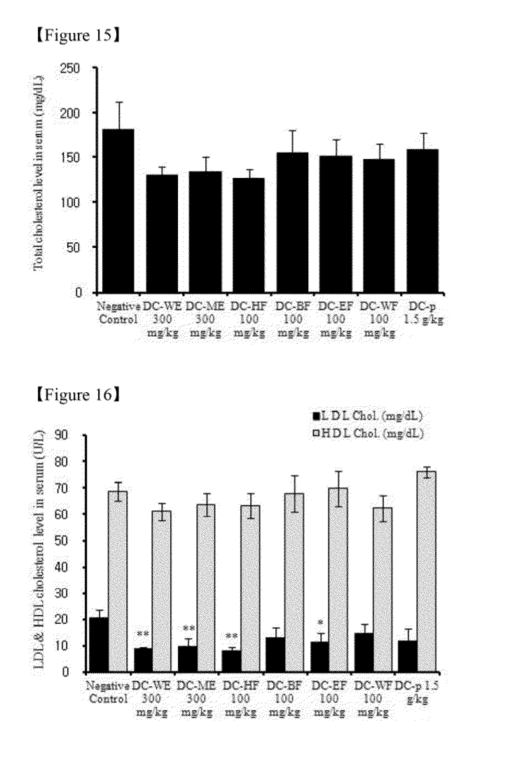

Serum samples were separated in the same manner as in 2-2 and serum total cholesterol levels were measured using an automatic serum analyzer. The results are shown in FIG. 15. The total cholesterol levels of the groups fed with DC-WE, DC-ME, DC-HF, DC-BF, DC-EF, DC-WF, and DC-p were lower than the total cholesterol level (181.5 mg/dL) of the non-treated group (NC), but there were no statistical significances among the groups.

2-4. Serum Low-Density Lipoprotein Cholesterol and High-Density Lipoprotein Cholesterol Changes

Serum samples were separated in the same manner as in 2-2 and serum low-density lipoprotein cholesterol and high-density lipoprotein cholesterol levels were measured using an automatic serum analyzer. The results are shown in FIG. 16. The low-density lipoprotein cholesterol levels of the groups fed with DC-WE, DC-ME, DC-HF, and DC-EF were statistically significantly lower than the low-density lipoprotein cholesterol level (20.7 mg/dL) of the non-treated group (NC) (p<0.05, p<0.01). From these results, it could be confirmed that the administration of the glycine soja extracts. DC-HF, and DC-EF were effective in decreasing low-density lipoprotein cholesterol, leading to improved blood circulation. In contrast, the high-density lipoprotein cholesterol levels of the groups fed with DC-WE, DC-ME, DC-HF, DC-BF, DC-EF, DC-WF, and DC-p were not substantially different from the high-density lipoprotein cholesterol level of the non-treated group.

2-5. Serum ALT and AST Changes

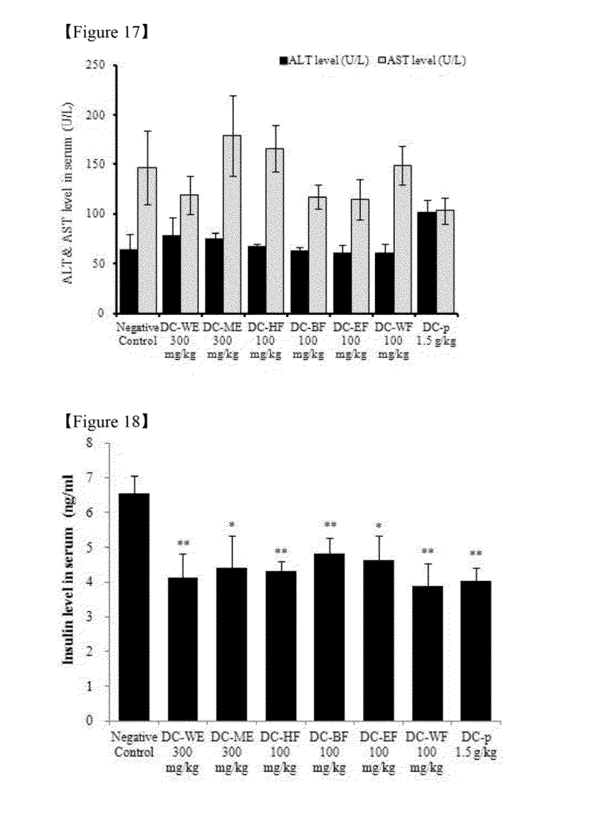

Serum samples were separated in the same manner as in 2-2 and serum ALT and AST levels were measured using an automatic serum analyzer. The results are shown in FIG. 17. The serum ALT and AST levels of the non-treated group were not substantially different from those of the groups fed with DC-WE, DC-ME, DC-HF, DC-BF, DC-EF, DC-WF, and DC-p. Therefore, no hepatotoxicity was observed in all experimental groups.

2-5, Serum Insulin Changes

Serum samples were separated in the same manner as in 2-2 and serum insulin levels were measured using a Mouse Insulin ELISA kit (SHIBAYAGI, Japan). The results are shown in FIG. 18. The serum insulin levels of the groups fed with DC-WE, DC-ME, DC-HF, DC-BF, DC-EF, DC-WF, and DC-p were 4.15 ng/ml, 4.43 ng/ml, 4.31 ng/ml, 4.83 ng/ml, 4.63 ng/ml, 3.89 ng/ml, and 4.15 ng/ml, respectively, which were statistically significantly lower (by at least 25%) than the serum insulin level (6.55 ng/ml) of the non-treated group (NC). Particularly, the insulin level of the groups fed with DC-WF was considerably low by 40% or more compared to that of the non-treated group. These results suggest that DC-WE, DC-WF, and DC-p have suppressive efficacy on blood glucose and effectively ameliorates resistance to insulin, which is a problem of type 2 diabetes mellitus.

2-6. Adipose Tissue Changes

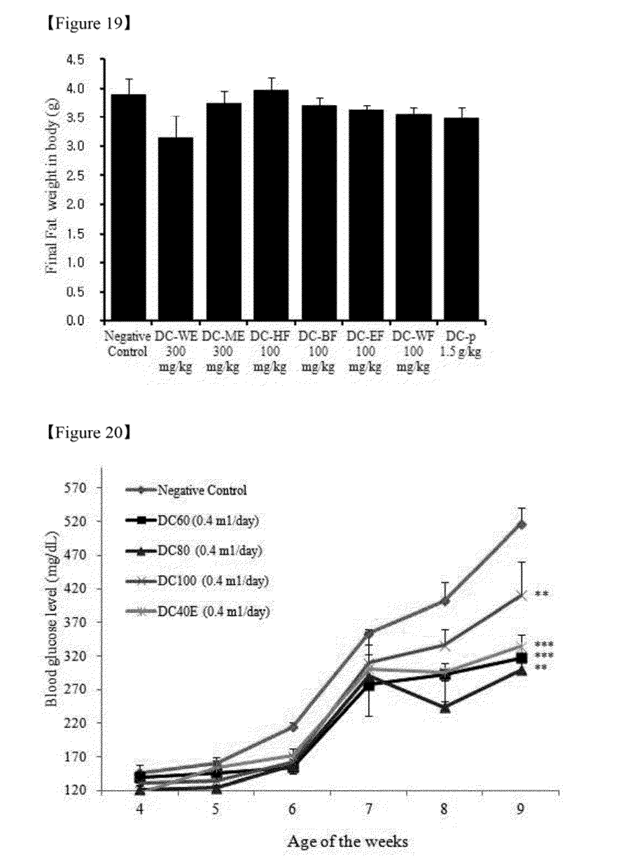

After the experiment was completed at the age of 10 weeks, abdominal, epididymal, and inguinal adipose tissues were excised from each db/db mouse. The total weight of the adipose tissues was measured. The results are shown in FIG. 19. The adipose tissue weight of the group fed with DC-WE was lower by about 19% or more than that of the non-treated group (NC), but there was no statistical significance between the two groups.

### Experimental Example 4

### Comparison of Antidiabetic Effects of the Glycine soja Extracts Obtained at Different Extraction Temperatures

1. Materials and Methods

1-1. Diabetes Mellitus Model Animals and Experimental Design

Male db/db mice, aged 3 weeks, were acclimatized to an animal breeding room for 1 week. 6 mice were assigned to each experimental group. Drug was administered orally in divided portions (each 0.2 ml) at 10 a.m. and 4 p.m. daily using a mouse sonde. The blood glucose levels of the animals were measured from the age of 4 weeks at intervals of 1 week for a total of 6 weeks. All animals were fasted for 6 h from 9 a.m. to 3 p.m. every Wednesday. Blood samples were collected from the mouse tail vein and blood glucose levels were measured using a portable blood glucose meter (OneTOUCH@Ultra, Johnson & Johnson, USA). The overall experimental design was the same as that in Experimental Example 2.

1-2. Test Materials and Administration Methods

The glycine soja extracts obtained at different extraction temperatures in Example 3 were used as test materials. Each of the test materials was suspended in physiological saline and administered orally in divided portions (each 0.2 ml) at 10 a.m. and 4 p.m. daily using a mouse sonde. The following are a total of 5 experimental groups used.

Test Group Classification
Control group (db/db mice) (n=4)
DC60 extract (0.4 ml/day, o.p) (n=4)
DC80 extract (0.4 ml/day, o.p) (n=4)
DC100 extract (0.4 ml/day, o.p) (n=4)
DC40E extract (0.4 ml/day, o.p) (n=4)

1-3. Measurement of Adipose Tissue Weights of db/db Mice with Type 2 Diabetes Mellitus

After administration of each DC extract for 6 weeks, abdominal, epididymal, and inguinal adipose tissues were excised from each experimental animal. The total weight of the adipose tissues was measured.

1-4. Biochemical Analysis of Plasma Lipids of db/db Mice with Type 2 Diabetes Mellitus

After each test material was administered for 6 weeks, blood glucose level was measured at the final 8^{th} week. 2 days after the measurement, an autopsy was conducted. For the autopsy, each animal was fasted for 12 h. Blood was drawn from the heart under ether anesthesia, put in a tube for serum separation, and centrifuged at 3000 rpm for 20 min to obtain serum, which was used as a sample for the analysis of biochemical indices. After plasma was separated, ALT and AST levels, which are liver function indices, and total cholesterol, high-density lipoprotein (HDL) cholesterol, low-density lipoprotein (LDL) cholesterol, and triglyceride levels, which are indices of lipid contents in plasma and liver, in the plasma were measured using an automatic biochemical analyzer (Hitachi-720, Hitachi Medical, Japan).

1-5. Serum Glucose and Insulin Analysis of db/db Mice with Type 2 Diabetes Mellitus

Serum was separated in the same manner as in 1-4. The levels of glucose and insulin in the serum were analyzed. Plasma was obtained as a sample in the same manner. The level of insulin in the plasma was measured using a Mouse Insulin ELISA kit (Shibayagi, Japan) and an ELISA reader (Labsystems, Finland).

1-6. Statistical Processing

Data obtained from the above experiments were expressed as mean±standard error and significance assay was determined using Student's T-test.

2. Results

2-1. Blood Glucose Changes

The DC60, DC80, DC100, and DC40E extracts were administered orally to 4-week-old db/db mouse models twice (in the morning and afternoon) a day for 5 weeks. Blood samples were drawn from the mouse tail vein and blood glucose levels were measured using a portable blood glucose meter once a week. All mice were fasted from 6 h before measurement. The results are shown in FIG. 20. The blood glucose level of the negative control (NC) steadily increased to 146.8 mg/dL at the age of 4 weeks, 160.5 mg/dL at the age of 5 weeks, 215.0 mg/dL at the age of 6 weeks, 353.8 mg/dL at the age of 7 weeks, and 401.8 mg/dL at the age of 8 weeks. The blood glucose level of the negative control at the final 9^{th} week was 516.5 mg/dL, which was 3.5 times higher than that at the age of 4 weeks. In contrast, the blood glucose levels of the groups fed with the DC extracts were statistically significantly lower than those of the NC (at least p<0.01).

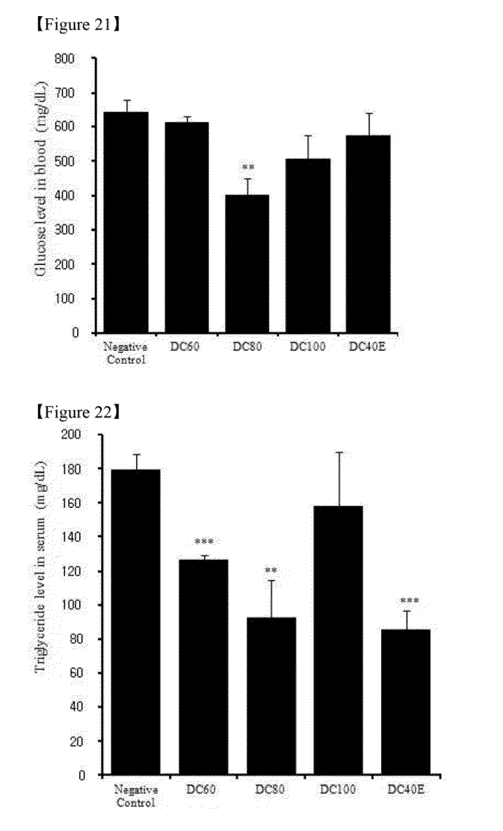

The blood glucose levels of the groups fed with the DC extracts were measured at the 9^{th} week. The results are shown in FIG. 21. The blood glucose levels of the groups fed with the DC60, DC80, DC100, and DC40E extracts were 317.0 mg/dl, 299.3 mg/dl, 410.3 mg/dl, and 334.0 mg/dl, which were lower by 38.6%, 42.0%, 20.5%, and 35.3% than the blood glucose level of the NC, respectively.

2-2. Serum Triglyceride (TG) Changes

The experiment was completed at the age of 9 weeks. All mice were fasted (for about 16 h) on the day before measurement and anesthetized with ethyl ether. Blood was drawn from the heart of each animal with a 3 ml syringe and left standing at room temperature for 1 h. Thereafter, the blood was centrifuged at 3000 rpm for 10 min to separate serum. Changes in serum triglyceride (TG) level were measured using an automatic serum analyzer. The results are shown in FIG. 22. The serum triglyceride (TG) level of the non-treated group was 179.0 mg/dL and the serum glucose levels of the groups fed with DC60, DC80, DC100, and DC40E were 126.8 mg/dL, 92.3 mg/dL, 158.3 mg/dL, and 85.5 mg/dL, respectively. The serum glucose levels of the groups fed with the DC extracts except the DC100 extract were statistically significantly lower (by at least 29.0%) than the serum glucose level of the non-treated group (p<0.01, p<0.001). Particularly, the triglyceride levels of the groups fed with the DC60 and DC40E extracts were 50% or less of the triglyceride level of the non-administered group, demonstrating that the DC60 and DC40E extracts had improving effects on blood circulation.

2-3. Serum Total Cholesterol Changes

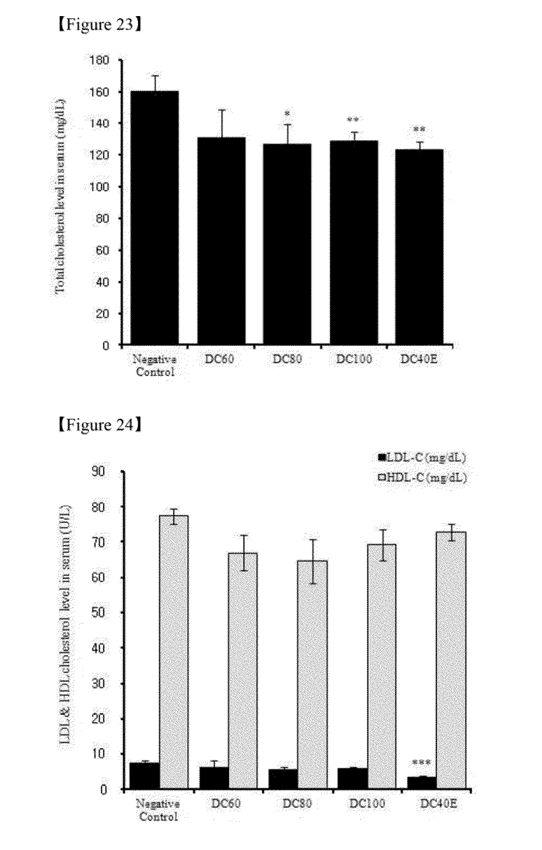

Serum samples were separated in the same manner as in 2-2 and serum total cholesterol levels were measured using an automatic serum analyzer. The results are shown in FIG. 23. The total cholesterol levels of the groups fed with DC80, DC80, and DC40E were statistically significantly lower than the total cholesterol level (160.3 mg/dL) of the non-treated group. In contrast, the total cholesterol level of the group fed with DC60 was slightly lower than that of the non-treated group, but there was no statistical significance between the two groups.

2-4. Serum Low-Density Lipoprotein Cholesterol and High-Density Lipoprotein Cholesterol Changes

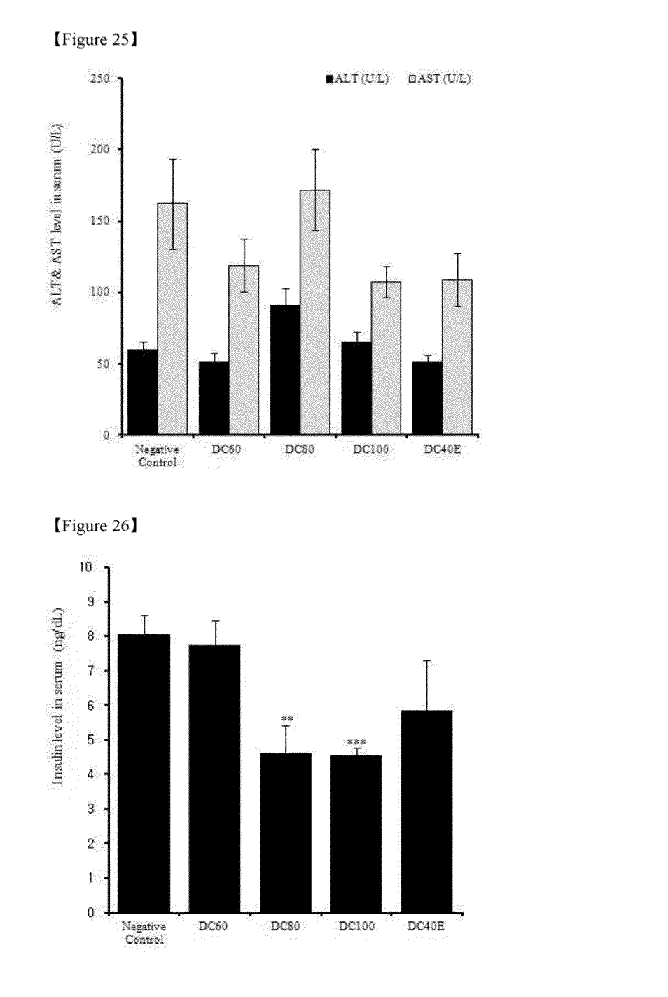

Serum samples were separated in the same manner as in 2-2 and serum low-density lipoprotein cholesterol and high-density lipoprotein cholesterol levels were measured using an automatic serum analyzer. The results are shown in FIG. 24. The low-density lipoprotein cholesterol level of the group fed with DC40E was statistically significantly lower (by 54% or more) than the low-density lipoprotein cholesterol level (7.4 mg/dL) of the non-treated group (p<0.001). In contrast, the low-density lipoprotein cholesterol levels of the groups fed with DC60, DC80, and DC100 were lower than the low-density lipoprotein cholesterol level of the non-treated group, but there were no statistical significances among the groups. From these results, it could be confirmed that the administration of DC40E was effective in decreasing low-density lipoprotein cholesterol level, leading to improved blood circulation. In contrast, the high-density lipoprotein cholesterol levels of the groups fed with DC60. DC80, DC 100, and DC40E were not substantially different from the high-density lipoprotein cholesterol level of the non-treated group.

2-5. Serum ALT and AST Changes

Serum samples were separated in the same manner as in 2-2 and serum ALT and AST levels were measured using an automatic serum analyzer. The results are shown in FIG. 25. The serum ALT and AST levels of the non-treated group were not substantially different from those of the groups fed with DC60. DC80, DC100, and DC40E. Therefore, no hepatotoxicity was observed in all experimental groups.

2-6. Serum Insulin Changes

Serum samples were separated in the same manner as in 2-2 and serum insulin levels were measured using a Mouse Insulin ELISA kit (SHIBAYAGI, Japan). The results are shown in FIG. 26. The serum insulin levels of the groups fed with DC60, DC80, DC100, and DC40E were 7.73 ng/ml, 4.59 ng/ml, 4.52 ng/ml, and 5.82 ng/ml, respectively. Particularly, the serum insulin levels of the groups fed with DC80 and DC 100 were statistically significantly lower (by at least 43%) than the serum insulin level (8.06 ng/ml) of the non-treated group (NC) (p<0.01). These results suggest that the administration of DC80 and DC100) has suppressive efficacy on blood glucose and effectively ameliorates resistance to insulin, which is a problem of type 2 diabetes mellitus.

2-7. Adipose Tissue Changes

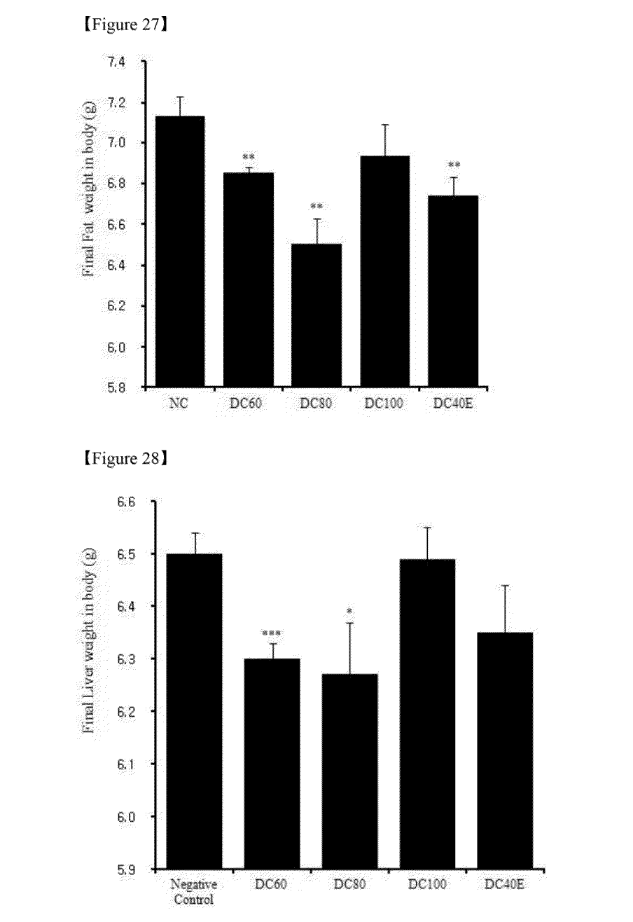

After the experiment was completed at the age of 10 weeks, abdominal, epididymal, and inguinal adipose tissues were excised from each db/db mouse. The total weight of the adipose tissues was measured. The results are shown in FIG. 27. The adipose tissue weights of the groups fed with DC60, DC80, and DC40E were statistically significantly lower than the adipose tissue weight of the non-treated group (NC) (p<0.01).

2-8. Liver Tissue Changes

After the experiment was completed at the age of 10 weeks, liver tissues were excised from each db/db mouse. The total weight of the liver tissues was measured. The results are shown in FIG. 28. The liver tissue weights of the groups fed with DC60 and DC80 were statistically significantly lower than the liver tissue weight of the non-treated group (NC) (p<0.001, p<0.05, respectively).

### Experimental Example 5

### Efficacy Comparison and Assay Test

The efficacies of the glycine soja extracts, the extracts obtained at different extraction temperatures, and anthocyanins, pinitol, and banaba/Cr complex, which are known as antidiabetic active substances derived from plants in the Zingiberaceae family or other natural products were compared and assayed in BKS.Cg-m^{+/+} Lepr^{db/J} homozygous diabetic (db/db) mice.

1. Materials and Methods

1-1. Test Materials and Comparative Test Materials

1) Test Groups

“DC5 extract” and “DC25 extract” were obtained in the same manner as in Example 3, except that extraction temperatures were changed to 5° C. and 25° C., respectively. 100 g of the glycine soja water extract (DC60 extract) prepared at an extraction temperature of 60° C. in Example 3 was dissolved in 2 L of water, passed through a D101 column to obtain Fraction 1, and then 2 L of 30% ethanol was passed through the D101 column to obtain Fraction 2. Fractions 1 and 2 were concentrated using a rotary evaporator under reduced pressure at 45° C. to obtain DC60-1 and DC60-2, respectively.

2) Comparative Test Groups

“Anthocyanins”: An extract containing 30% of anthocyanins isolated and purified from glycine max.

“Pinitol”: Product containing 95% or more of pinitol (Sigma-Aldrich)

“Banaba/Cr complex”: Wellness Banaba Gold Chrome™ (containing 400 mg/g banaba leaf extract, 200 mg/g indigestible maltodextrin and 0.1 mg/g chromium)

1-2. Diabetes Mellitus Model Animals and Experimental Design

Male db/db mice, aged 3 weeks, were acclimatized to an animal breeding room for 1 week. 4 mice were assigned to each experimental group. Drug was administered orally in divided portions (each 0.2 ml) at 10 a.m. and 4 p.m. daily using a mouse sonde. The blood glucose levels, food intakes, and body weight changes of the animals were measured from the age of 4 weeks at intervals of 1 week for a total of 6 weeks. All animals were fasted for 6 h from 9 a.m. to 3 p.m. every Wednesday. Blood samples were collected from the mouse tail vein and blood glucose levels were measured using a portable blood glucose meter (OneTOUCH@Ultra, Johnson & Johnson, USA). The overall experimental design was the same as that in Experimental Example 2.

1-3. Administration Methods and Experimental Group Classification

Each of the test materials was suspended in physiological saline and administered orally in divided portions (each 0.2 ml) at 10 a.m. and 4 p.m. daily using a mouse sonde. The following are a total of 8 experimental groups used.

Classification of Experimental Groups
NC: Control group (db/db mice, aged 6 weeks) (n=4)
DC60-1 (100 mg/kg, o.p) (n=4)
Anthocyanins (100 mg/kg, o.p) (n=4)
DC25 (100 mg/kg, o.p) (n=4)
Pinitol (100 mg/kg, o.p) (n=4)
DC60-2 (100 mg/kg, o.p) (n=4)
DC5 (100 mg/kg, o.p) (n=4)
BANABA: (300 mg/kg, o.p) (n=4)

1-4. Measurement of Adipose Tissue Weight

After the drug (test material) was administered to each experimental group for 6 weeks, abdominal, epididymal, and inguinal adipose tissues were excised from each experimental animal. The total weight of the adipose tissues was measured.

1-5. Biochemical Analysis of Plasma Lipids

After each test material was administered for 6 weeks, blood glucose level was measured at the final 8^{th} week. 2 days after the measurement, an autopsy was conducted. For the autopsy, each animal was fasted for 12 h. Blood was drawn from the heart under ether anesthesia, put in a tube for serum separation, and centrifuged at 3000 rpm for 20 min to obtain serum, which was used as a sample for the analysis of biochemical indices. After plasma was separated, the levels of total cholesterol, high-density lipoprotein (HDL) cholesterol, low-density lipoprotein (LDL) cholesterol, triglyceride, BUN, and phospholipid in the plasma were measured using an automatic biochemical analyzer (Hitachi-720, Hitachi Medical, Japan).

1-6. Serum Glucose and Insulin Analysis

Serum was separated in the same manner as in 1-5. The levels of glucose and insulin in the serum were analyzed. Plasma was obtained as a sample in the same manner. The plasma insulin level was measured using a Mouse Insulin ELISA kit (Shibayagi, Japan) and an ELISA reader (Labsystems, Finland).

1-7. Statistical Processing

Data obtained from the above experiments were expressed as mean±standard error and significance assay was determined using Student's T-test.

2. Results

2-1. Blood Glucose Changes

BANABA (300) mg/kg/day) and the other samples (each 100 mg/kg/day) were administered orally to 4-week-old db/db mouse models twice (in the morning and afternoon) a day for 6 weeks. Blood samples were drawn from the mouse tail vein and blood glucose levels were measured using a portable blood glucose meter once a week. All mice were fasted from 6 h before measurement. The results are shown in FIG. 29. The blood glucose level of the non-treated group (NC) steadily increased to 113.5 mg/dL at the age of 4 weeks, 216.5 mg/dL at the age of 5 weeks, 303.8 mg/dL at the age of 6 weeks, 448 mg/dL at the age of 7 weeks, and 467.5 mg/dL at the age of 8 weeks. The blood glucose level of the negative control at the final 9^{th} week was 484.8 mg/dL, which was 4.2 times higher than that at the age of 4 weeks. In contrast, the blood glucose levels of all test groups and comparative test groups were statistically significantly lower than those of the NC (at least p<0.01). At the 9^{th} week, the blood glucose levels of the groups fed with Pinitol, BANABA, and Anthocyanins as comparative test groups were 231.8 mg/dL, 316.8 mg/dL, and 356.3 mg/dL, which were lower by 52.1%, 34.6%, and 26.5% than the blood glucose level (about 485 mg/dL) of the NC, respectively. The blood glucose levels of the groups fed with DC25, DC5. DC60-1, and DC60-2 as test groups were 253.8 mg/dL, 316.8 mg/dL, and 359.3 mg/dL, and 302.5 mg/dL, which were lower by 47.6%, 34.6%, 25.8%, and 38.6% than the blood glucose level of the NC, respectively. In conclusion, all test groups and comparative test groups were found to have hypoglycemic effects at the final 9^{th} week, and their efficacies decreased in the order: Pinitol (52.1%)>DC25 (47.6%)>DC60-2 (38.6%)>DC5 (34.6%)=BANABA (34.6%)>Anthocyanins (26.5%)=DC60-1 (25.8%).

2-2. Body Weight, Food Intake, and Food Efficiency Ratio

During administration of the drugs (test materials) for 6 weeks, changes in the body weight of the test groups over the control group were measured. The results are shown in FIG. 30. Changes in the body weight of the groups fed with DC60-1, DC25, Pinitol, and DC60-2 were observed. Specifically, the body weights of 1-2 animals in each experimental group were much higher or lower than the average body weight. The food intakes were measured and the results are shown in Table 1. After completion of the tests, the daily food intakes of the groups fed with DC60-1, DC25, and Pinitol were observed to be smaller than the daily food intake of the non-treated group (NC). The food intakes and body weight gains were used to calculate the food efficiency ratios of the groups for 5 weeks. As a result, statistically significant differences compared with the non-treated group (NC) were found in the groups fed with DC25 (p<0.05), DC60-2 (p<0.05), and BANABA (p<0.05). In addition, the food efficiency ratios (FER, %) of the groups fed with DC25 and Pinitol were similar to the PER of the non-treated group (NC), the food efficiency ratios of the groups fed with DC25, Anthocyanins, and BANABA were at least 20% lower than the PER of the non-treated group (NC), and the food efficiency ratios of the groups fed with DC60-1 and DC60-2 were at least 28% lower than the PER of the non-treated group (NC).

TABLE 1

| | Food | Body | Food |
|---|---|---|---|
| | intake | weight gain | efficiency ratio |
| Group | (g/day) | (g/day) | (PER, %) |
| Negative control (NC) | 5.45 | 0.661 ± 0.05 | 12.1 ± 0.88 |
| DC60-1 | 4.93 | 0.429 ± 0.09 | 8.69 ± 1.77 |
| Anthocyanins | 5.47 | 0.518 ± 0.09 | 9.47 ± 1.71 |
| DC25 | 4.70 | 0.468 ± 0.03 | 9.95 ± 0.64 |
| Pinitol | 4.20 | 0.500 ± 0.05 | 11.9 ± 1.28 |
| DC60-2 | 5.14 | 0.429 ± 0.07 | 8.34 ± 1.45 |
| DC5 | 5.37 | 0.443 ± 0.15 | 11.0 ± 0.55 |
| BANABA | 5.95 | 0.561 ± 0.02 | 9.70 ± 0.34 |

2-3. Serum Triglyceride (TG) Changes

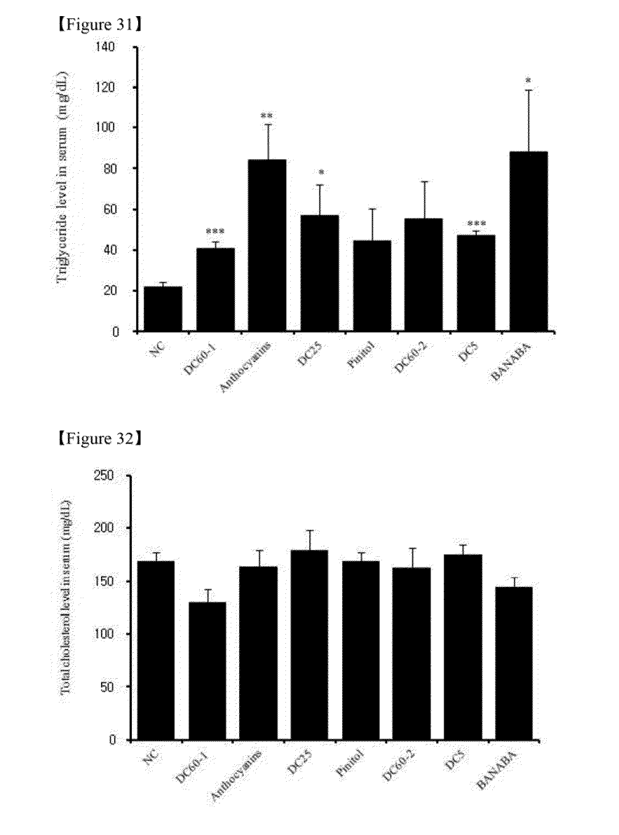

The experiment was completed at the age of 9 weeks. All mice were fasted (for about 16 h) on the day before measurement and anesthetized with ethyl ether. Blood was drawn from the heart of each animal with a 3 ml syringe and left standing at room temperature for 1 h. Thereafter, the blood was centrifuged at 3000 rpm for 10 min to separate serum. Changes in serum triglyceride level were measured using an automatic serum analyzer. The results are shown in FIG. 31. The serum triglyceride (TG) levels of the groups fed with DC60-1, DC60-2, DC25, DC5, and Pinitol were approximately twice as high as the serum triglyceride level (22 mg/dL) of the non-treated group (NC). The triglyceride (TG) levels of the groups fed with Anthocyanins and BANABA were about 4 times higher than the serum triglyceride level of the NC.

2-3. Serum Total Cholesterol Changes

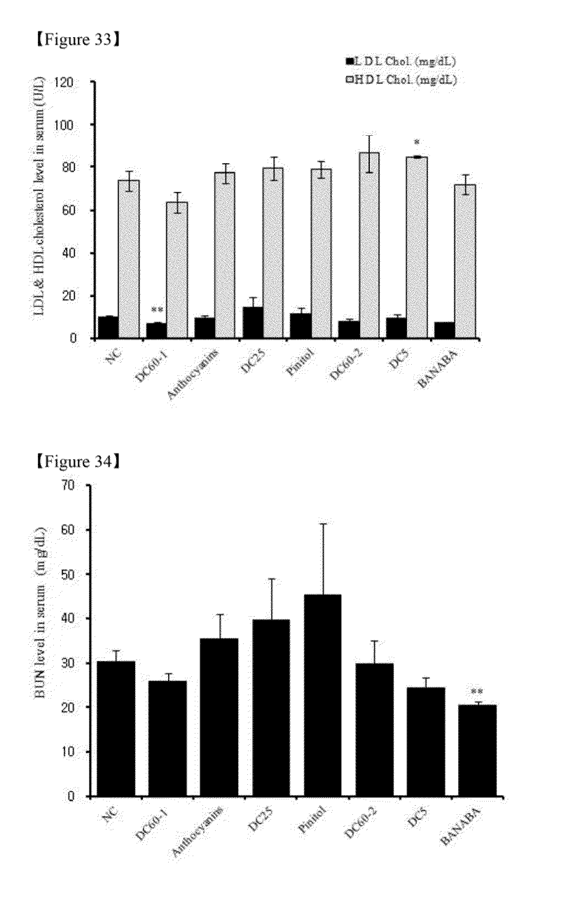

Serum samples were separated in the same manner as in 2-2 and serum total cholesterol levels were measured using an automatic serum analyzer. The results are shown in FIG. 32. The total cholesterol levels of the groups fed with DC60-1 and BANABA were lower than the total cholesterol level (168.3 mg/dL) of the non-treated group (NC), but there were no statistical significances among the groups. The serum total cholesterol levels of the groups fed with DC6-2, DC25, DC5, Anthocyanins and Pinitol were not substantially different from the serum total cholesterol level of the NC.

2-4. Serum Low-Density Lipoprotein Cholesterol and High-Density Lipoprotein Cholesterol Changes

Serum samples were separated in the same manner as in 2-2 and serum low-density lipoprotein cholesterol and high-density lipoprotein cholesterol levels were measured using an automatic serum analyzer. The results are shown in FIG. 33. The low-density lipoprotein cholesterol level of the group fed with DC60-1 decreased to about 65% of the low-density lipoprotein cholesterol level (10.1 mg/dL) of the non-treated group, with a statistical significance (p<0.01). The low-density lipoprotein cholesterol levels of the groups fed with DC60-2 and BANABA were lower than the low-density lipoprotein cholesterol level of the non-treated group, but there were no statistical significances among the groups. In contrast, the low-density lipoprotein cholesterol levels of the groups fed with DC25, DC5, Anthocyanins, Pinitol, and BANABA were not substantially different from the low-density lipoprotein cholesterol level of the non-treated group. The high-density lipoprotein cholesterol levels of all test groups were not substantially different from the high-density lipoprotein cholesterol level of the non-treated group.

2-5. Serum BUN Changes

Serum samples were separated in the same manner as in 2-2 and serum BUN levels were measured using an automatic serum analyzer. The results are shown in FIG. 34. Diabetes mellitus may be a factor affecting the level of BUN in serum. A decrease in the quantity of muscles resulting from obesity may vary the level of BUN secreted from muscles, etc. The BUN level of No. 4 mouse of the Pinitol-administered group was 92.8 mg/dL, which was 3 times or more higher than that (30.2 mg/dL) of the non-treated group. Therefore, the administration of Pinitol cannot rule out the possibility of nephrotoxicity. The serum BUN levels of the groups fed with DC60-1, DC60-2, DC25, DC5, Anthocyanins, and BANABA were not substantially from the serum BUN level of the non-treated group (NC).

2-6. Serum Insulin Changes

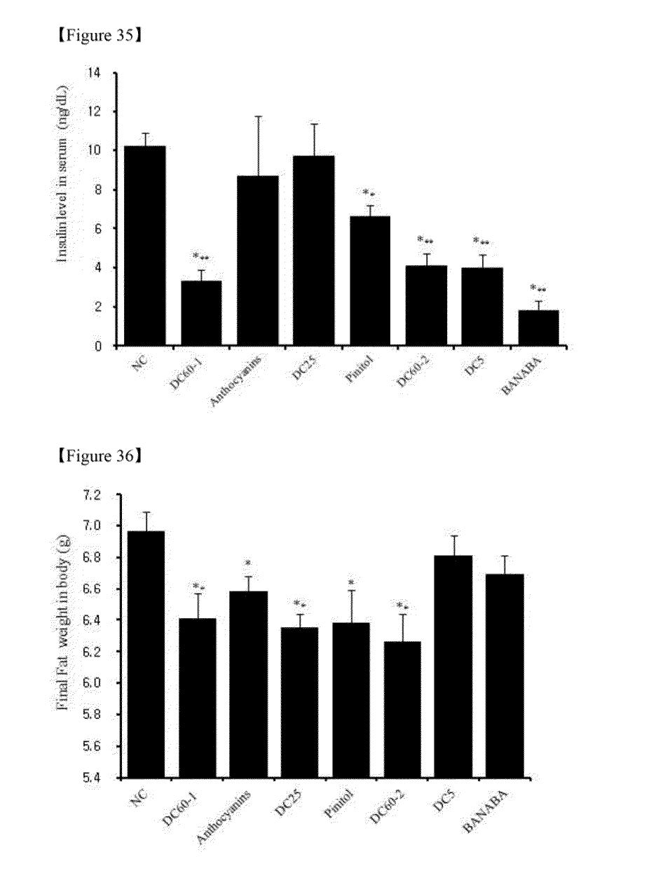

Serum samples were separated in the same manner as in 2-2 and serum insulin levels were measured using a Mouse Insulin ELISA kit (SHIBAYAGI, Japan). The results are shown in FIG. 35. The serum insulin levels of the groups fed with DC60-1, DC60-2, DC5, and BANABA were 3.3 ng/ml, 4.1 ng/ml, 4.0 ng/ml, and 1.8 ng/ml, respectively, which were statistically significantly lower (by at least 60%) than the serum insulin level (10.2 ng/ml) of the non-treated group (NC) (p<0.001). The insulin level of the group fed with Pinitol was 6.6 ng/ml, which was statistically significantly lower (by 35.3%) than that of the NC (p<0.01). The serum insulin levels of the groups fed with DC25 and Anthocyanins were not significantly lowered compared with the serum insulin level of the NC. Finally, the suppressive efficacies of the test groups on insulin resistance decreased in the order: BANABA (82.4%)>DC60-1 (67.6%)>DC5 (60.9%)>DC60-2 (59.8%)>Pinitol (35.3%).

2-7. Adipose Tissue Changes

After the experiment was completed at the age of 10 weeks, abdominal, epididymal, and inguinal adipose tissues were excised from each db/db mouse. The total weight of the adipose tissues was measured. The results are shown in FIG. 36. The adipose tissue weights of the groups fed with DC60-1, Anthocyanins. DC25, Pinitol, and DC60-2 were statistically significantly lower (by about 5.4%) than the adipose tissue weight of the non-treated group (NC) (p<0.05, p<0.01). However, the adipose tissue weights of the groups fed with DC5 and BANABA were not substantially different from the adipose tissue weight of the NC.

2-8. Liver Tissue Changes

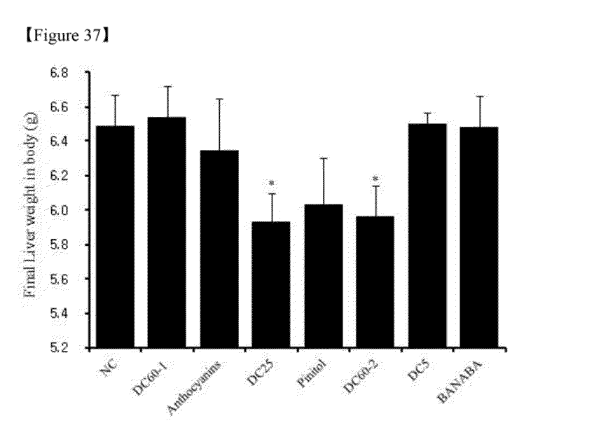

After the experiment was completed at the age of 10 weeks, liver tissues were excised from each db/db mouse. The total weight of the liver tissues was measured. The results are shown in FIG. 37. The liver tissue weights of the groups fed with DC25, Pinitol, and DC60-2 were statistically significantly lower (by about 7.0%) than the liver tissue weight of the non-treated group (NC) (p<0.05). However, the liver tissue weights of the groups fed with DC60-1, Anthocyanins. Pinitol, DC5, and BANABA administered groups were not substantially different from the liver tissue weight of the NC.

### Experimental Example 6

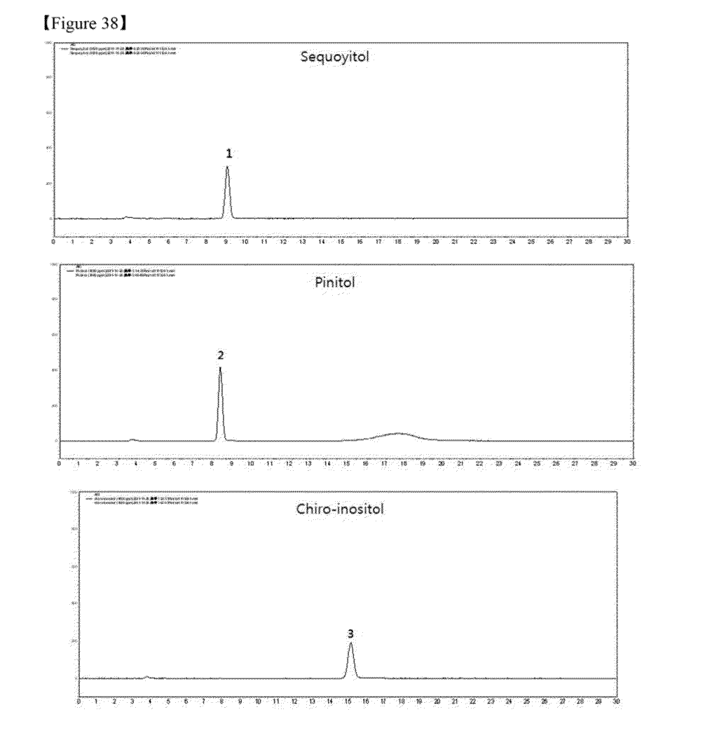

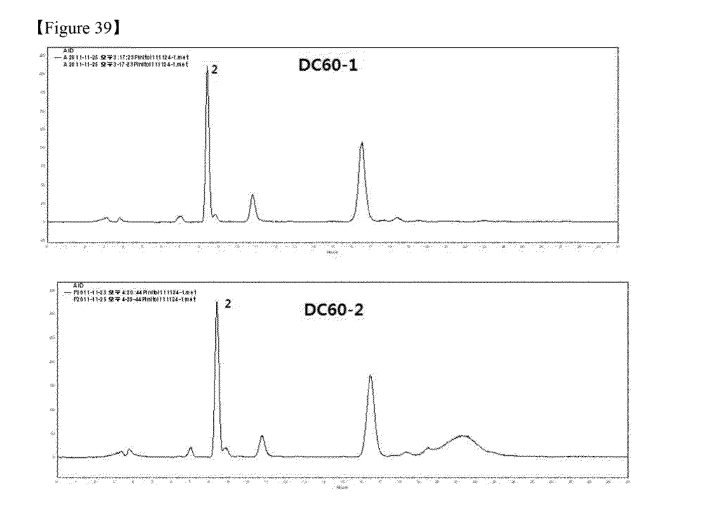

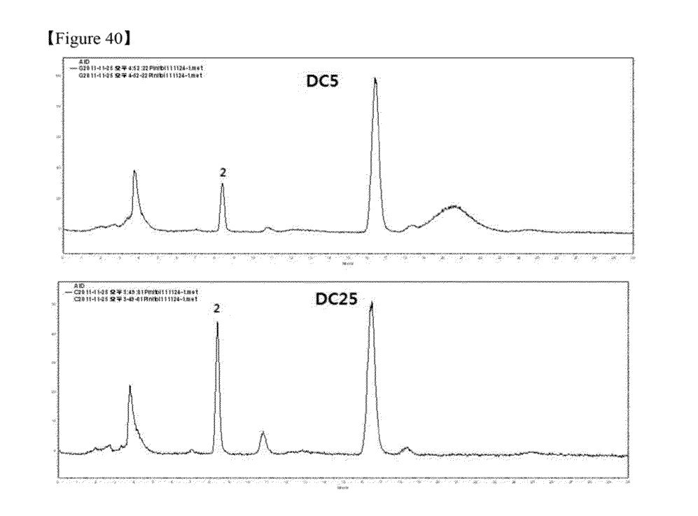

The test materials DC60-1, DC60-2, DC5. DC25 used in Experimental Example 5 were analyzed in order to directly confirm whether a major active ingredient of the glycine soja extracts is sequoyitol or pinitol, which are known as antidiabetic active substances derived from plants in the Zingiberaceae family, and to determine the contents of sequoyitol, pinitol, and chiro-inositol in the glycine soja extracts. The analysis conditions are as follows. The analysis results are shown in FIGS. 38-40 and Table 2. Neither sequoyitol nor chiro-inositol was detected in DC60-1, DC60-2, DC5, and DC25. A slight amount of pinitol was detected, but the hypoglycemic effects of the test materials were not proportional to the pinitol content (see 2-1 of Experimental Example 5 and FIG. 29).

Analysis Conditions

System: HITACHI Elite Lachrom HPLC

Detector: ELSD Detector

Column: NH₂ column (5 μm, 4.6×250 mm)

Column temperature: 40° C.

Flow rate: 1.0 ml/min

Injection volume: 10 μl

Solvents: Acetonitrile 85%:H₂O 15% (Isocratic)

TABLE 2

| Test material | Pinitol | Sequoyitol | Chiro-inositol |
|---|---|---|---|
| DC60-1 | 6.3% | Not detected | Not detected |
| DC60-2 | 10.7% | Not detected | Not detected |
| DC5 | 1.2% | Not detected | Not detected |
| DC25 | 1.5% | Not detected | Not detected |

### Example 4

Hereinafter, exemplary formulations of pharmaceutical compositions including the extracts according to the present invention will be explained. However, these formulations do not serve to limit the invention and are set forth for illustrative purposes only.

Formulation Example 1: Preparation of powder

| | |
|---|---|
| DC-WE extract | 20 mg |
| Lactose | 100 mg |
| Talc | 10 mg |

The above ingredients were mixed together and filled in an airtight bag to prepare a powder formulation.

Formulation Example 2: Preparation of tablet

| | |
|---|---|
| DC-WE extract | 10 mg |
| Corn starch | 100 mg |
| Lactose | 100 mg |
| Magnesium stearate | 2 mg |

After the above ingredients were mixed together, the mixture was compressed into tablets in accordance with a suitable method known in the art.

Formulation Example 3: Preparation of capsule

| | | |
|---|---|---|
| DC-WE extract | 10 | mg |
| Crystalline cellulose | 3 | mg |
| Lactose | 14.8 | mg |
| Magnesium stearate | 0.2 | mg |

In accordance with a suitable method known in the art, the ingredients were mixed together and filled in a gelatin capsule to prepare a capsule formulation.

Formulation Example 4: Preparation of injectable formulation

| | |
|---|---|
| DC-WE extract | 10 mg |
| Mannitol | 180 mg |
| Sterile distilled water for injection | 2974 mg |
| Na₂HPO₄12H₂O | 26 mg |

In accordance with a suitable method known in the art, the above ingredients were filled in an ampoule (2 ml) to prepare an injectable formulation.

Formulation Example 5: Preparation of liquid formulation

| | | |
|---|---|---|
| DC-WE extract | 20 | mg |
| Isomerized sugar | 10 | g |
| Mannitol | 5 | g |

| | |
|---|---|
| Purified water | Appr. amount |

A liquid formulation was prepared in accordance with a suitable method known in the art. First, the above ingredients were dissolved in purified water, and then an appropriate amount of lemon flavor was added thereto. The ingredients were mixed and the mixture was made up to a total of 100 ml with purified water. The resulting mixture was filled in an amber glass bottle and sterilized to prepare a liquid formulation.

1. A pharmaceutical composition for the prevention and treatment of diabetes mellitus or diabetic complications comprising a heat-treated powder or extract of glycine soja as an active ingredient.

2. The pharmaceutical composition according to claim 1, wherein the heat-treated powder is obtained by heat treating glycine soja or a glycine soja powder at 40 to 100° C.

3. The pharmaceutical composition according to claim 1, wherein the heat-treated powder is obtained by steaming glycine soja or a glycine soja powder at 60 to 90° C.

4. The pharmaceutical composition according to claim 1, wherein the extract is obtained by extracting a glycine soja powder at 100° C. or less.

5. The pharmaceutical composition according to claim 1, wherein the extract is obtained by extracting a glycine soja powder with water, an organic solvent having 1 to 4 carbon atoms, or a mixture thereof at 0 to 90° C.

6. The pharmaceutical composition according to claim 1, wherein the extract is a fraction obtained by extracting a glycine soja powder and fractionating the extract with water or an organic solvent having 1 to 4 carbon atoms.

7. The pharmaceutical composition according to claim 1, wherein the diabetic complications are arteriosclerosis, cerebral infarction, cerebral thrombosis, myocardial infarction, hypertension, hyperlipidemia, and obesity.

8. A food composition for the prevention and amelioration of diabetes mellitus or diabetic complications comprising a heat-treated powder or extract of glycine soja as an active ingredient.

9. The food composition according to claim 8, wherein the heat-treated powder is obtained by steaming glycine soja or a glycine soja powder at 40 to 100° C.

10. The food composition according to claim 8, wherein the heat-treated powder is obtained by steaming glycine soja or a glycine soja powder at 60 to 90° C.

11. The food composition according to claim 8, wherein the extract is obtained by extracting a glycine soja powder at 100° C. or less.

12. The food composition according to claim 8, wherein the extract is obtained by extracting a glycine soja powder with water, an organic solvent having 1 to 4 carbon atoms, or a mixture thereof at 0 to 90° C.

13. The food composition according to claim 8, wherein the extract is a fraction obtained by extracting a glycine soja powder and fractionating the extract with water or an organic solvent having 1 to 4 carbon atoms.

14. The food composition according to claim 8, wherein the diabetic complications are arteriosclerosis, cerebral infarction, cerebral thrombosis, myocardial infarction, hypertension, hyperlipidemia, and obesity.

15. The food composition according to claim 8, wherein the food is a health functional food having a formulation selected from tablets, capsules, powders, granules, liquids, and pills.

16. The food composition according to claim 8, wherein the food is selected from beverages, powdered beverages, solid foods, chewing gums, teas, vitamin complexes, and food additives.
